SEQUENCE LISTING (2 sequences)

SEQ ID NO: 1 - DNA, 24 bp, Artificial Sequence
  Chemically synthesized oligonucleotide (6-FAM labeled forward primer)
tgacattttagggagctggagaca

SEQ ID NO: 2 - DNA, 20 bp, Artificial Sequence
  Chemically synthesized oligonucleotide (unlabeled reverse primer)
acaaagtctggccataggac

(19) United States
(12) Patent Application Publication
(10) Pub. No.: US 2013/0287747 A1
(43) Pub. Date: Oct. 31, 2013

(54) Methods of Treating Acute Kidney Injury Using Mesenchymal Stem Cells

(71) Applicant: ALLOCURE, INC., Burlington, MA (US)

(72) Inventors: Dena Minning, Indialantic, FL (US); Robert M. Brenner, Weston, MA (US)

(21) Appl. No.: 13/873,863

(22) Filed: Apr. 30, 2013

Related U.S. Application Data
(60) Provisional application No. 61/640,226, filed on Apr. 30, 2012.
(60) Provisional application No. 61/645,298, filed on May 10, 2012.

(51) Int. Cl. A61K 35/28; A61K 45/06

(52) U.S. Cl. CPC A61K 35/28; A61K 45/06
USPC 424/93.21; 424/93.7

(57) ABSTRACT

The invention relates to methods of treating acute kidney injury (AKI) in a patient by administering a therapeutic amount of mesenchymal stem cells (MSC) to a patient in need thereof. Administration of MSCs ameliorates AKI in the patient when administered up to at least 48 hours following kidney injury or decline in kidney function.

### RELATED APPLICATIONS

This application claims the benefit of priority under 35 U.S.C. §119(e) to U.S. Patent Application Ser. No. 61/640,226 filed Apr. 30, 2012, and U.S. Patent Application Ser. No. 61/645,298 filed May 10, 2012, each of which are hereby incorporated by reference in their entireties.

### INCORPORATION-BY-REFERENCE OF SEQUENCE LISTING

The instant application contains a Sequence Listing which has been submitted via EFS-Web. The contents of the text file named “38447-507001US_ST25.txt”, which was created on Apr. 30, 2013 and is 1 KB in size, are hereby incorporated by reference in their entirety.

### FIELD OF THE INVENTION

The present invention generally relates to the use of mesenchymal stem cells to treat acute kidney injury.

### BACKGROUND OF THE INVENTION

Stem cell therapy offers a promising new option for the treatment of human disease. Adult stem cells have been used successfully to treat patients in various clinical trials across a number of clinical conditions. Mesenchymal stem cells (MSCs) are bone marrow, adipose, and/or cord blood derived cells that have the ability to differentiate into a variety of cell types under certain conditions, possess immunomodulatory properties and secrete chemokines, cytokines and growth factors (Schinkothe et al., Stem Cells Dev. 2008; 17: 199-206), together making them ideal candidate therapies of various disorders (Porada et al., Curr Stem Cell Res Ther. 2006; 1:365-9). MSCs have been used successfully to treat a number of conditions in animal models and are currently being evaluated in clinical trials to treat different diseases including acute kidney injury (AKI), myocardial infarction, graft versus host disease, Crohn's disease and others (Giordano et al., J Cell Physiol. 2007; 211: 27-35).

MSCs are effective in reducing kidney injury and enhancing recovery of kidney function in animal models of AKI, including an ischemia/reperfusion model as well as in cytotoxicity models such as a cisplatin toxicity model. Importantly, in these models, MSC do not or only rarely directly contribute to differentiated kidney cell types, e.g. tubular cells or endothelial cells (Humphreys et al. Minerva Urol Nefrol. 2006; 58: 329-37). Instead, MSCs mediate benefit and promote kidney recovery through paracrine and endocrine mechanisms via the release of secreted mediators including stromal cell-derived factor-1 (SDF-1), vascular endothelial growth factor (VEGF) and other vasculotropic factors, insulin-like growth factor (IGF-1) (Imberti et al., J Am Soc Nephrol. 2007; 18: 2921-8), epidermal growth factor (EGF) (Tögel et al. Am J Physiol Renal Physiol. 2007; 292: F1626-35) and other factors that promote organ repair. Of note, the beneficial effect of MSCs has been reproduced using MSC conditioned medium in an animal model of AKI. (Bi et al. J Am Soc Nephrol. 2007; 18: 2486-96).

AKI is a serious medical condition associated with deleterious consequences, including the need for acute dialysis, extended length of hospital stay, increased mortality and development of chronic kidney disease with the attendant risk of end-stage kidney disease. Unfortunately, the unmet need in AKI is critical as there are no approved therapies, and clinical management is limited to supportive measures.

Thus, there is a need in the art for additional effective therapies for AKI.

### SUMMARY OF THE INVENTION

The invention provides methods of treating AKI in a subject by administering a therapeutically effective amount of MSCs (e.g., from a human or a non-human animal) to a subject (e.g., a human or non-human animal) in need thereof up to at least 48 hours following kidney injury and/or decline in kidney function, wherein the MSCs ameliorate AKI in the subject.

Those skilled in the art will recognize that a decline in kidney function can be measured by a number of methods, including, but not limited to, an increase in serum creatinine (SCr) level of at least 0.3 mg/dL. For example, the increase in serum creatinine (SCr) levels can be at least 0.5 mg/dL or between 0.3 mg/dL and 0.5 mg/dL. Decline in kidney function can also be further measured by an increase in one or more additional serum/blood biomarkers and/or an increase in one or more urine biomarkers. By way of non-limiting example, the one or more additional serum/blood biomarkers may be selected from blood urea nitrogen (BUN), Cystatin C, and/or Beta-trace protein (BTP) (also known as Prostaglandin D Synthase).

Those skilled in the art will also recognize that the one or more urine biomarkers may be selected from Podocalyxin, Nephrin, Alpha 1-microglobulin, Beta 2-microglobulin, Glutathione S-transferase, Interleukin-18, Kidney Injury Molecule-1 (KIM-1), Liver-Type Fatty Acid-Binding Protein, Netrin-1, Neutrophil Gelatinase-Associated Lipocalcin (NGAL), and N-Acetyl-Beta-D-Glucosaminidase (NAG).

Thus, a decline in kidney function can be measured by an increase in serum creatinine (SCr) alone or in combination with an increase in one or more biomarkers selected from blood urea nitrogen (BUN), Cystatin C, Beta-trace protein (BTP) (also known as Prostaglandin D Synthase), Podocalyxin, Nephrin, Alpha 1-microglobulin, Beta 2-microglobulin, Glutathione S-transferase, Interleukin-18, Kidney Injury Molecule-1 (KIM-1), Liver-Type Fatty Acid-Binding Protein, Netrin-1, Neutrophil Gelatinase-Associated Lipocalcin (NGAL), and/or N-Acetyl-Beta-D-Glucosaminidase (NAG). The decline in kidney function can also be measured by an increase in one or more serum/blood biomarkers (e.g. SCr, BUN, Cystatin C, and/or BTP (also known as Prostaglandin D Synthase)) and/or an increase in one or more urine biomarkers (e.g., Podocalyxin, Nephrin, Alpha 1-microglobulin, Beta 2-microglobulin, Glutathione S-transferase, Interleukin-18, KIM-1, Liver-Type Fatty Acid-Binding Protein, Netrin-1, NGAL, and/or NAG). In some embodiments, the decline in kidney function is measured by an increase in one or more of SCr, BUN, Cystatin C, BTP (also known as Prostaglandin D Synthase, Podocalyxin, Nephrin, Alpha 1-microglobulin, Beta 2-microglobulin, Glutathione S-transferase, Interleukin-18, KIM-1, Liver-Type Fatty Acid-Binding Protein, Netrin-1, NGAL, and/or NAG.

Those skilled in the art will recognize that the invention also provides methods of treating AKI in a subject by administering a therapeutically effective amount of MSCs to a patient in need thereof up to at least 48 hours following a clinical diagnosis of AKI in the patient, wherein the MSCs ameliorate AKI in the patient. A clinical diagnosis of AKI may be made using any method known in the art, including, but not limited to, the methods of measuring a decline in kidney function described herein.

Also provided are human mesenchymal stem cells (hMSC) for use in a method of treating acute kidney injury (AKI) in a subject, wherein the hMSCs are for administration to the subject up to at least 48 hours following a decline in kidney function of the subject, wherein the decline in kidney function is measured by an increase in serum creatinine level of at least 0.3 mg/dL.

In some embodiments, the therapeutically effective amount of MSCs is between about 7×10⁵ and about 15×10⁶ cells/kg, e.g., about 7×10⁵ cells/kg, about 2×10⁶ cells/kg, about 5×10⁶ cells/kg, about 7×10⁶ cells/kg, about 10×10⁶ cells/kg, or about 15×10⁶ cells/kg. For example, the therapeutically effective amount of MSCs is about 2×10⁶ cells/kg to about 5×10⁶ cells/kg.

The patient may suffer from or be at high risk of suffering from or be suspected of suffering from an acute deterioration in kidney function (e.g., renal excretory function, control of volume, endocrine function, and/or any other kidney function affected by AKI).

In various embodiments, the MSCs are administered to the patient at the time of onset of kidney injury and/or the decline in kidney function, at least 24 hours following kidney injury and/or the decline in kidney function, at least 48 hours following kidney injury and/or the decline in kidney function, between 24 and 48 hours following kidney injury and/or the decline in kidney function, between the onset of and 24 hours following kidney injury and/or the decline in kidney function, or at any point in between (e.g., 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, or 48 hours following kidney injury and/or the decline in kidney function).

The MSCs can be administered to the patient using any route of administration known in the art. By way of non-limiting example, the MSCs can be administered intra-arterially or intravenously to the patient. In some embodiments, the MSCs are administered to the patient in a biologically and physiologically compatible solution. Preferably, the solution is not enriched for pluripotent hematopoietic stem cells.

The MSCs can be autologous or allogeneic cells. Additionally, the MSCs can be non-transformed stem cells. Moreover, the patient may be any living organisms such as humans, non-human animals (e.g., monkeys, cows, sheep, horses, pigs, cattle, goats, dogs, cats, mice, or rats), cultured cells therefrom, and transgenic species thereof.

The MSCs are expanded in vitro to produce an enriched population of human MSCs. Any expansion method known in the art can be used to produce the enriched population.

In addition, the MSCs can be obtained from any source known in the art. For example, in one embodiment, the MSCs are isolated from bone marrow aspirates and adhere to a plastic culture dish while substantially all other cell types remain in suspension. In other embodiments, the MSCs are obtained from a bone marrow sample, from a cryopreserved sample, from a Master Cell Bank (MCB), and/or from any other source known to those skilled in the art.

By way of non-limiting example, the MSCs are expanded in a platelet lysate (PL) supplemented culture medium. Those skilled in the art will recognize that MSCs that have been cultured in PL supplemented culture media will express Prickle 1 at a higher degree than MSCs that have been cultured in fetal bovine serum (FBS) supplemented culture media. For example, the population of human MSCs expresses Prickle 1 to an eight-fold higher degree than MSCs that have been cultured in FBS supplemented culture media. (See, e.g., Lange et al., Cellular Therapy and Transplantation 1:49-53 (2008), which is herein incorporated by reference in its entirety). Those skilled in the art will recognize that a population of human MSCs that has been cultured in platelet lysate may be less immunogenic than MSCs that have been cultured in fetal calf serum supplemented culture media. Moreover the use of PL instead of FBS supplemented culture media reduces infectious risk and overall safety and regulatory concerns associated with the use of FBS.

Human MSCs suitable for use in the methods of the invention preferably have 32 or fewer GT repeats in both alleles of the human heme oxygenase (HO-1) promoter region. For example, the human MSCs utilized may have two short alleles, two medium alleles, or one short and one medium allele in the HO-1 promoter region, wherein a short allele has ≦26 GT repeats in the HO-1 promoter region and wherein a medium allele has between 27 and 32 GT repeats in the HO-1 promoter region. MSCs containing one or more long alleles are less therapeutically effective. Therefore, ideally, the human MSCs do not have any long alleles, wherein a long allele has >32 GT repeats in the HO-1 promoter region.

As used herein, a “short” allele can have ≦26 GT repeats in the HO-1 promoter region (e.g., between about 21 and about 26 GT repeats); a “medium” allele can have between about 27 and about 32 GT repeats in the HO-1 promoter region; and a “long” allele can have >32 GT repeats in the HO-1 promoter region (e.g., between about 33 and about 44 GT repeats).

Those skilled in the art will recognize that the number of GT repeats in an allele of the HO-1 promoter region can be analyzed using any suitable method known in the art, including, but not limited to Fragment Length Analysis and DNA sequencing methodologies.

In some embodiments, the MSCs are genetically modified, to augment the renoprotective potency of said cells prior to administration to the patient.

In further embodiments, the invention involves also delivering a therapeutic amount of a stimulant of human MSC mobilization to the patient, wherein the stimulant mobilizes stem cells to the kidney.

In some embodiments, the patient or subject suffers from or is at high risk of suffering from or developing an acute deterioration in kidney function. In addition or alternatively, the patient or subject in need thereof has undergone cardiac surgery. For example, a decline in kidney function occurs in the patient 48 hours or less following the cardiac surgery and/or following the patient's removal from cardiopulmonary bypass. The type of cardiac surgery can include, but is not limited to, coronary artery bypass grafting, valve surgery, and/or any other surgery utilizing cardiopulmonary bypass. “Subjects in need thereof” can include subjects who experience kidney injury and/or a decline in kidney function within 6 days, 4 days, 48 hours, 24 hours, or 12 hours of cardiac surgery. Preferably, a subject in need thereof is one who experiences kidney injury and/or a decline in kidney function within 48 hours of cardiac surgery.

In any of the methods described herein, the MSCs can be pre-differentiated in vitro prior to administration to the patient. By way of non-limiting example, the MSCs are pre-differentiated into endothelial cells and/or into renal tubular cells.

### BRIEF DESCRIPTION OF THE DRAWINGS

FIG. 1 is a graph showing that rat MSC (rMSC) treatment significantly reduced serum creatinine (SCr) in the bilateral ischemia-reperfusion (I/R) AKI rat model of human AKI. SCr data (mg/dL) are expressed as a means±standard error of the mean (SEM). Two-way analysis of variance (ANOVA) analysis using JMP statistical software was conducted to assess differences between means for each rMSC-treated group compared to the vehicle-treated group.

FIG. 2A is a graph showing that rMSC treatment reduced SCr area under the curve (AUC) in the bilateral I/R AKI rat model. FIG. 2B is a graph showing the SCr AUC decreased by up to 40% after treatment with rMSC. In FIGS. 2A and 2B, Group A=vehicle, Group B=rMSC administered at 0 hours after I/R, Group C=rMSC administered at 24 hours after I/R, Group D=rMSC administered at 48 hours after I/R. SCr AUC data (mg day/dL) are expressed as means±SEM. Two-way ANOVA analysis using JMP software was conducted to assess differences between means for each rMSC-treated group compared to the vehicle-treated group.

FIG. 3 is a graph showing that rMSC treatment significantly reduced blood urea nitrogen (BUN) in the bilateral AKI rat model. BUN concentration (mg/dL) data are expressed as means±SEM. Two-way ANOVA analysis using JMP software was conducted to assess differences between means for each rMSC-treated group compared to the vehicle-treated group.

FIG. 4A is a graph showing the rMSC treatment reduced BUN AUC in the bilateral I/R AKI rat model. FIG. 4B is a graph showing the BUN AUC decreased by up to 35% after treatment with rMSC. In FIGS. 4A and 4B, Group A=vehicle, Group B=rMSC administered at 0 hours after I/R, Group C=rMSC administered at 24 hours after I/R, Group D=rMSC administered at 48 hours after I/R. BUN AUC data (mg day/dL) are expressed as means±SEM. Two-way ANOVA analysis using JMP software was conducted to assess differences between means for each rMSC-treated group compared to the vehicle-treated group.

FIGS. 5A and 5B are photographs showing renal injury in a vehicle-treated rat versus a rat treated with rMSC at 0 h post-reperfusion, respectively, in the bilateral I/R AKI rat model (Magnification 20×). Low (20×) magnification photomicrographs from vehicle-treated rat (FIG. 5A; Rat 4) and a rat treated with rMSCs at 0 hours post-reperfusion (FIG. 5B; Rat 25). Note the increased number and prominence of dilated tubules at the corticomedullary junction and extending into the cortex in the vehicle-treated rat (5A). The grading for this lesion was marked in vehicle-treated rat 4 (5A) and slight in rMSC-treated rat 25 (5B).

FIG. 6 is a series of photographs showing renal injury in vehicle-treated rats versus rats treated with rMSC at 0 hours post-reperfusion in the bilateral I/R AKI rat model (magnification 50×). Representative photomicrographs (magnification 50×) from vehicle-treated rats (6A; Rat 4 and 6B; Rat 11) and rats treated with rMSCs at 0 hours post-reperfusion (6C; Rat 19 and 6D; Rat 25) are shown. Note the increased proportion of the cortex containing affected parenchyma in the vehicle-treated rats compared to that of the rMSC-treated rats. Additionally, tubules in the vehicle-treated rats were replaced by tubular regeneration and exhibited mineralization and/or proteinuria. The grading for this lesion was marked in vehicle-treated rats 4 and 11 (FIGS. 6A and B) and slight in rMSC-treated rats 19 and 25 (FIGS. 6C and D).

FIG. 7 is a series of photographs showing renal injury in vehicle-treated rats versus rats treated with rMSC at 0 hours post-reperfusion in the bilateral I/R AKI rat model (magnification 100×). Representative photomicrographs (magnification 100×) from vehicle-treated rats (7A; Rat 4 and 7B; Rat 11) and rats treated with rMSCs at 0 hours post-reperfusion (7C; Rat 19 and 7D; Rat 25) are shown. Note numerous dilated and/or mineralized tubules, tubular proteinosis, and replacement of the interstitum with tubular regeneration in vehicle-treated rats (FIGS. 7A and B). The grading for this lesion was marked in vehicle-treated rats 4 and 11 (FIGS. 7A and B) and slight in rMSC-treated rats 19 and 25 (FIGS. 7C and D).

### DETAILED DESCRIPTION OF THE INVENTION

The details of one or more embodiments of the invention have been set forth in the accompanying description below. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural references unless the context clearly dictates otherwise. All patents and publications cited in this specification are incorporated by reference in their entirety.

For convenience, certain terms used in the specification, examples and claims are collected here. Unless otherwise defined, all technical and scientific terms used herein have the same meaning as commonly understood by one of ordinary skill in the art to which this invention pertains.

As used herein, the terms “patient,” “individual,” “subject”, “host”, or the like are used interchangeably herein to refer to either a human or a non-human animal.

MSC are a promising biologic therapy being developed for the prevention and treatment of AKI. MSC have effectively ameliorated AKI in a variety of preclinical models, including the rat bilateral renal I/R, mouse cisplatin, and rat glycerol models. In prior rat AKI studies, MSC were administered prophylactically or up to 24 hours after I/R.

A blinded, placebo-controlled study was conducted in the bilateral renal I/R AKI model in male Sprague-Dawley rats. The objective of this study was to evaluate the ability of allogeneic rat MSC (rMSC) to ameliorate AKI in the rat bilateral renal I/R model when administered at 0, 24 and 48 hours post-injury (e.g., following decline in kidney function). As described in Example 1, infra, AKI was induced in male Sprague-Dawley rats by bilateral renal I/R. All animals were treated intra-arterially with either vehicle or allogeneic rMSC (5×10⁶ cells/kg) immediately after reperfusion (0 hours), and 24 and 48 hours post-reperfusion, respectively (n=12/group).

SCr and BUN were measured at baseline, and at 24, 48, 72, 96, and 120 hours post-I/R. Animals were sacrificed at 120 hours, and kidney pathology was assessed.

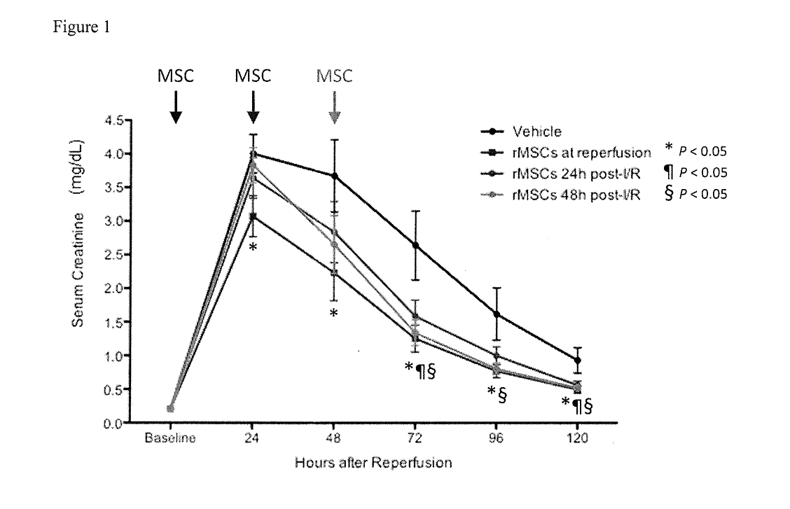

Treatment with rMSC at 0, 24, or 48 hours post-I/R abrogated AKI (see FIG. 1). SCr of animals treated with rMSC at 0 hours was significantly decreased at all time points after MSC treatment (i.e., 24, 48, 72, 96 and 120 hours), compared to vehicle treated animals (P<0.05). SCr of animals treated with rMSC at 24 hours was significantly decreased at 72 and 120 hours compared to vehicle-treated animals (P<0.05) and was less than vehicle-treated animals at 96 hours (P=0.05). Animals treated with rMSC at 48 hours exhibited significantly lower SCr at all time points after rMSC treatment (72, 96 and 120 hours) compared to vehicle-treated animals (P<0.05). The SCr area under the curve (AUC) was similarly reduced. Serum BUN and BUN AUCs showed similar results to those observed for SCr. In addition, rMSC treatment was associated with diminished severity of pathologic lesions and lower tubular epithelial degeneration/necrosis scores, compared to vehicle treatment.

These data demonstrate that allogeneic rMSC effectively attenuate AKI when administered up to 48 hours after I/R (e.g., after decline of kidney function).

Assaying MSCs for Therapeutic Effectiveness or Potency

MSCs can be evaluated for their therapeutic effectiveness or potency. The number of GT repeats in the HO-1 promoter region of MSCs may be indicative of the therapeutic efficacy of the MSCs. Analyzing the number of GT repeats in both donor alleles (whether obtained from a cryopreserved MSC sample, from fresh blood, from a Master Cell Bank and/or from other suitable genetic material), helps to determine whether the MSC population is enriched to be robust, and, thus, be therapeutically effective.

Preferably, the number of GT repeats in both HO-1 alleles is not too long. Indeed, as described herein, MSCs having fewer GT repeats in both HO-1 alleles express higher HO-1 protein levels and are more likely to be therapeutically effective.

A (GT)n repeat region that can decrease transcription is located between −190 and −270 of the human HO-1 promoter and is absent in the mouse HO-1 gene. (See Sikorski et al. at page F429). In addition, DNA length polymorphisms of this region vary between human subjects and correlate with activity of various diseases, such as emphysema, coronary artery disease, and other disorders. Typically, individuals with shorter repeats (<25) demonstrate higher induced HO-1 protein levels and milder disease manifestations, whereas individuals with longer repeats have lower HO-1 levels and more severe disease. (See Sikorski et al., Am J Physiol Renal Physiol 286:F424-F441 (2004); Zarjou et al., Am J Physiol Renal Physiol 300:F254-F262 (2011); Exner et al., Free Radical Biology & Medicine 37(8):1097-104 (2004), which are herein incorporated by reference in their entireties).

As used herein, the term “short allele” refers to MSC HO-1 alleles having ≦26 GT repeats in the human HO-1 promoter region.

As used herein, the term “medium allele” refers to MSC HO-1 alleles having between 27 and 32 GT repeats in the human HO-1 promoter region.

As used herein, the term “long allele” refers to MSC HO-1 alleles having >32 GT repeats in the human HO-1 promoter region.

Studies in mice have demonstrated that HO-1 is essential for their therapeutic potential in cisplatin-induced AKI. (See Zarjou et al., Am J Physiol Renal Physiol 300:F254-F262 (2011)). Moreover, the absence of HO-1 expression in MSCs limit their protective paracrine effects including the angiogenic potential of MSCs and for growth factor and/or reparative factor secretion and expression by MSC. (See Zarjou et al. at p. F260).

Moreover, the number of GT repeats in the HO-1 promoter region of any nucleated cell of the human body may be measured by any method known in the art. For example, Fragment Length Analysis can be used. Briefly, PCR is used to amplify fragments from both HO-1 alleles per cell using PCR primers that flank the HO-1 promoter region containing the GT repeats. The resulting PCR fragments are separated on a column and the “predicted” sizes are reported (in base pairs). Fragment Length Analysis is, thus, able to report relative size differences between different alleles. The absolute size of the PCR fragments can subsequently be determined using methods well known to those of ordinary skill in the relevant art.

Fragment Length Analysis (see Exner et al., Free Radical Biology & Medicine 37(8):1097-104 (2004)) is used to determine the number of GT repeats. Briefly, PCR is used to amplify fragments from both HO-1 alleles per MSC using PCR primers, one of which is fluorescently labeled, that flank the HO-1 promoter region containing the GT repeats. The resulting PCR fragments are separated on a column (for example, at an external vendor), and the “predicted” sizes are reported (in base pairs).

Fragment Length Analysis is a commonly used method for determining the length of FAM-labeled PCR fragments. However, fragment length analysis only predicts the relative size of different fragments and the relative differences between different alleles. Based upon the fragment length data, it is believed that a PCR fragment size of 302 base pairs corresponds to 23 GT repeats. However, those skilled in the art will appreciate that the apparent fragment length could differ on a different column.

In accordance with the methods of the instant invention, donors or MSCs will be excluded if they have one or more long GT repeat alleles. Thus, only those donors or MSCs having two short alleles, two medium alleles, or one medium and one short allele will be accepted. Only MSCs without a long allele will be used clinically.

According to certain embodiments of the invention, other MSC markers are also measured. For example, the presence of CD105 and/or CD90 is measured in some embodiments. In other embodiments, the absence of CD34 and/or CD45 is measured. The presence of CD 105 and/or CD90 as well as the absence of CD34 and/or CD45 is indicative of the MSC phenotype. In other embodiments, adipogenic, osteogenic and/or chondrogenic assays are used to show that the MSCs possess the characteristic ability of trilineage differentiation.

Mesenchymal Stem Cells Cultured in Platelet Lysate (PL) Supplemented Media

MSCs may be passaged or expanded according to any methods known in the art. For example, published PCT application WO2010/017216 and US patent publication US20110293576, which are incorporated herein by reference in their entireties, describe methods for the culture and expansion of MSCs in platelet lysate (PL) supplemented media.

The invention provides MSCs with unique properties that make them particularly beneficial for use in the treatment of kidney pathology. The MSCs of the invention are grown in media containing PL, as described in greater detail below. The culturing of MSCs in PL-supplemented media creates MSCs that are more protective against ischemia-reperfusion damage than MSCs grown in FBS.

The MSCs of the invention, cultured in PL-supplemented media constitute a population with (i) surface expression of antigens such as CD 105, CD90, CD73, CD44, and MHC I, but lacking hematopoietic markers such as CD45, CD34 and CD14; (ii) preservation of the multipotent trilineage (osteoblasts, adipocytes and chondrocytes) differentiation capability after expansion with PL, however the adipogenic differentiation was delayed and needed longer times of induction. This decreased adipogenic/lipogenic ability is a favorable property because in mice the intra-arterial injection of MSCs for treatment of kidney injury has revealed formation of adipocytes (Kunter et al., J Am Soc Nephrol 2007 June; 18(6):1754-64). These results are reflected in the gene expression profile of PL-generated cells revealing a down-regulation of genes involved in fatty acid metabolism, described in greater detail below.

The MSCs of the invention, cultured in PL-supplemented media have been described to act immunomodulatory by impairing T-cell activation without inducing anergy. There is a dilution of this effect in vitro in mixed lymphocyte cultures (MLC) leading eventually to an activation of T-cells if decreasing amounts of MSCs, not cultured in PL-supplemented media, are added to the MLC reaction. This activation process is not observed when PL-generated MSCs are used in the MLC as a third party, as shown in greater detail below. It was concluded that the MSCs of the invention, cultured in PL-supplemented media are less immunogenic and that growing MSCs in FBS-supplemented media may act as a strong antigen or at least has adjuvant function in T-cell stimulation. This result again is reflected in differential gene expression showing a down-regulation of MHC II molecules verifying the decreased immunostimulation by MSC, as shown below.

Moreover, the MSCs of the invention, cultured in PL-supplemented media show up-regulation of genes involved in the cell cycle (e.g. cyclins and cyclin dependent kinases) and the DNA replication and purine metabolism when compared to MSCs cultured in FBS-supplemented media. On the other hand, genes functionally active in cell adhesion/extracellular matrix (ECM)-receptor interaction, differentiation/development, TGF-β signaling and TSP-I induced apoptosis could be shown to be down-regulated in the MSCs of the invention, cultured in PL-supplemented media when compared to MSCs cultured in FBS-supplemented media, again supporting the results of faster growth and accelerated expansion.

The MSCs of the invention, cultured in PL-supplemented media, when administered (e.g., intra-arterially) lead to improvement of repair and regeneration of injured tissue by ameliorating local inflammation, decreasing apoptosis, and by delivering growth factors and other mediators needed for the repair and/or regeneration of the damaged cells. Injured cells or organs secrete SDF-1 that draws MSCs to the site of injury through the chemokine receptor 4 (CXCR4).

The MSCs of the invention, cultured in PL-supplemented media are particularly good candidates for regenerative therapy in central nervous system (CNS) damage. They express the gene Prickle 1 to an eight-fold higher degree compared to MSCs cultured in FBS-supplemented media which is involved in neuroregeneration. Mouse Prickle 1 and Prickle 2 are expressed in postmitotic neurons and promote neuronal outgrowth (Okuda et al., FEBS Lett. 2007 Oct. 2; 581(24):4754-60). Furthermore, MAG (Myelin-associated glycoprotein) is expressed at 13-fold lower levels in the MSCs of the invention, cultured in PL-supplemented media. MAG is a cell membrane glycoprotein and may be involved in myelination during nerve regeneration. The lack of recovery after CNS injury is caused, in part, by myelin inhibitors including MAG. MAG acts as a neurite outgrowth inhibitor for most neurons tested but stimulates neurite outgrowth in immature dorsal root ganglion neurons (Vyas et al., Proc Natl Acad Sci USA, 2002; 99(12):8412-7). These differentially regulated genes would favor the use of PL cultured hMSC for regeneration of neuronal injury.

Additionally, the expression of retinoic acid receptor (RAR) responsive gene TIG1, shows 12 fold higher expression in the MSCs of the invention, cultured in PL-supplemented media) (Liang et al. Nature Genetics 2007; 39(2):178-188), Keratin 18 (9 fold higher expression in the MSCs of the invention, cultured in PL-supplemented media) (Bühler et al, Mol Cancer Res. 2005; 3(7):365-71), CRBP1 (cellular retinol binding protein 1, 5.7 fold higher expression in the MSCs of the invention cultured in PL-supplemented media) (Roberts et al., DNA Cell Biol. 2002; 21(1):1 1-9.) and Prickle 1 suggest a less tumorigenic phenotype of the MSCs of the invention, cultured in PL-supplemented media.

Furthermore, MSCs grown in PL-supplemented medium are more protective against ischemia-reperfusion damage than MSCs grown in FBS-supplemented medium.

Methods of Producing Mesenchymal Stem Cells

In certain embodiments, the mesenchymal stem cells (MSCs) of the invention are cultured in media supplemented with PL or FBS. In one embodiment of the method of producing MSCs of the invention, the starting material for the MSCs is bone marrow isolated from healthy donors. Preferably, these donors are mammals. More preferably, these mammals are humans. In one embodiment of the method of producing MSCs of the invention, the bone marrow is cultured in tissue culture cell factories between 2 and 10 days (e.g., 2, 3, 4, 5, 6, 7, 8, 9, or 10 days) prior to washing non-adherent cells from the cell factory. Optionally, the number of days of culture of bone marrow cells prior to washing non-adherent cells is 2 to 3 days. Preferably the bone marrow is cultured in PL containing media. 25-125 mL (e.g., 25, 30, 35, 40, 45, 50, 55, 60, 65, 70, 75, 80, 85, 90, 95, 100, 105, 110, 115, 120, or 125 mL) of bone marrow aspirate is cultured in 400-1500 mL (e.g., 400, 450, 500, 550, 600, 650, 700, 750, 800, 850, 900, 950, 1000, 1050, 1100, 1150, 1200, 1250, 1300, 1350, 1400, 1450, or 1500 mL) of PL supplemented media in a multi layered cell factory or other adequate tissue culture vessels, automated closed system bioreactors, or suspension bead technology (including enough media volume for each culture vessel technology).

After washing away the non-adherent cells, the adherent cells are also cultured in media that has been supplemented with PL or FBS. Thrombocytes (platelets) are a well-characterized human product already widely used clinically for patients in need. Platelets are known to produce a wide variety of factors, e.g. PDGF-BB, TGF-β, IGF-1, and VEGF. In one embodiment of the method of producing MSCs of the invention, an optimized preparation of PL is used. This optimized preparation of PL is made up of pooled platelet rich plasma (PRP) from at least 10 (e.g., about 10 to about 100; for example, about 10, about 15, about 20, about 25, about 30, about 35, about 40, about 45, about 50, about 55, about 60, about 65, about 75, about 80, about 85, about 90, about 95, or about 100) donors with a minimal concentration of 3×10⁹ thrombocytes/mL.

According to preferred embodiments of the method of producing MSCs of the invention, PL was prepared either from pooled thrombocyte concentrates designed for human use or from 7-13 (e.g., 7, 8, 9, 10, 11, 12, or 13) pooled buffy coats after centrifugation with 200×g for 20 min. Preferably, the PRP was aliquoted into small portions, frozen at −80° C., and thawed immediately before use. Thawing of PRP causes lysis of thrombocytes, generating PL, and release of growth factors that facilitate robust MSC growth. Multiple freeze and thaw cycles may increase the potency of the PL. PL-containing medium is prepared freshly for each lot production. In a preferred embodiment, medium contained αMEM (minimum essential medium alpha) as basic medium supplemented with 5 IU Heparin/mL and 2-10% (e.g., 2, 3, 4, 5, 6, 7, 8, 9, or 10%) of freshly thawed PL, which can be used for up to 28 days (e.g., 1, 2, 3, 4, 5, 6, 7, 8, 9, 10, 11, 12, 13, 14, 15, 16, 17, 18, 19, 20, 21, 22, 23, 24, 25, 26, 27, or 28 days) without significant loss of MSC growth supporting properties. The method of producing MSCs of the invention uses a method to prepare PL that differs from others according to the thrombocyte concentration and centrifugation forces. The composition of this PL is described in greater detail, below.

In one embodiment of the method of producing MSCs, the adherent cells are cultured in PL-supplemented media at 37° C. with approximately 5% CO₂ under hypoxic conditions. Preferably, the hypoxic conditions are an atmosphere of 5% O₂. In some situations hypoxic culture conditions allow MSCs to grow more quickly. This allows for a reduction of days needed to grow the cells to 90-100% confluence. Generally, it reduces the growing time by three days. In another embodiment of the method of producing MSCs of the invention, the adherent cells are cultured in PL-supplemented media at 37° C. with approximately 5% CO₂ under normoxic conditions, i.e. wherein the O₂ concentration is the same as atmospheric O₂, approximately 20.9%. Preferably, the adherent cells are cultured between 9 and 12 days (e.g., 9, 10, 11, or 12 days), being fed every 3-5 days (e.g., 3, 4, or 5 days) with PL-supplemented media. In one embodiment of the method of producing MSCs of the invention, the adherent cells are grown to between 80 and 100% confluence. Preferably, once this level of confluence is reached, the cell monolayers are detached from the culture vessel enzymatically by using recombinant porcine trypsin. The detached cells in suspension are plated for subsequent culture. The process of successive detaching and plating of cells is called passage.

In certain embodiments, the population of cells that is isolated from the culture vessel is between 50-99% MSCs. In other embodiments, isolated MSCs are enriched in MSCs so that 25%, 30%, 35%, 40%, 45%, 50%, 55%, 60%, 65%, 70%, 75%, 80%, 85%, 90%, 95%, 96%, 97%, 98% or 99% of the cell population are MSCs. In other embodiments, the MSCs are greater than 95% of the isolated cell population.

Preferably, the MSCs used in any of the methods, compositions, and kits described herein are free of infectious agents. In some embodiments, the MSCs have undergone fewer than 30 population doublings and are cultured to 80 to 100% confluence. Moreover, using the various methods described herein, MSC cell viability should be greater or equal to 70% (e.g., 70%, 75%, 80%, 85%, 90%, 95% or greater viability).

In another embodiment of the method of producing MSCs of the invention, the cells are frozen after they are released from the tissue culture vessel. Freezing is performed in a step-wise manner in a physiologically acceptable carrier, 2 to 10% serum albumin (e.g., 2, 3, 4, 5, 6, 7, 8, 9, and/or 10%) and 2-10% DMSO (e.g., 2, 3, 4, 5, 6, 7, 8, 9, and/or 10%). Thawing is also performed in a step-wise manner. Preferably, when thawed, the frozen MSCs of the invention are diluted about 2-8 fold (e.g., 2, 3, 4, 5, 6, 7, or 8-fold) to reduce DMSO concentration. In some embodiments, frozen MSCs of the invention are thawed quickly at 37° C. and administered intravenously without any dilution or washings. Optionally, the cells are administered following any protocol that is adequate for the transplantation of hematopoietic stem cells (HSCs). Preferably, the serum albumin is human serum albumin (HSA).

In another embodiment of the method of producing MSCs of the invention, the cells are frozen in aliquots of 10⁴-10¹² cells in 10 to 20 mL (e.g., 10, 11, 12, 13, 14, 15, 16, 17, 18, 19, or 20 mL) of physiologically acceptable carrier and HSA. In another embodiment of the method of producing MSCs of the invention, the cells are frozen in aliquots of 10⁶-10⁸ cells in 10 to 20 mL (e.g., 10, 11, 12, 13, 14, 15, 16, 17, 18, 19, or 20 mL) of physiologically acceptable carrier and HSA. In another embodiment of the method of producing MSCs of the invention, the cells administered in a dose of 10⁶-10⁸ cells per kg of subject body weight, in 50-150 mL (e.g., 50, 55, 60, 65, 70, 75, 80, 85, 90, 95, 100, 105, 110, 115, 120, 125, 130, 135, 140, 145, or 150 mL) of physiologically acceptable carrier and HSA. If the cells are administered IV, the dose of cells may be included in up to 1 L of physiologically acceptable carrier and HSA.

Those skilled in the art will recognize that any cryopreservation protocol or process known in the art can be used to freeze the MSCs of the invention.

In one aspect of these embodiments, when a therapeutic dose is being assembled, the appropriate number of cryovials is thawed in order to prepare the appropriate number of cells for the therapeutic dose based on the patient's body weight. Any thawing protocol or process known in the art can be used to thaw the MSCs of the invention prior to administration. Preferably, the number of cryovials is chosen based on the weight of the patient. The vials are thawed in a water bath and placed in a sterile infusion bag with 2-10% HSA (e.g., 2, 3, 4, 5, 6, 7, 8, 9, and/or 10%). Once in the bag, the MSCs do not aggregate and viability remains greater than 70% even when the MSCs are stored at room temperature for at least 8 hours. This provides ample time to administer the MSCs of the invention to a patient. Optionally, the physiologically acceptable carrier is Plasma-lyte A. Preferably the HSA is present at a concentration of 5-10% (e.g., 5, 6, 7, 8, 9, and/or 10%) w/v. Suspending the 10⁶-10⁸ cells MSCs of the invention in greater than 50 mL of physiological carrier is critical to their biological activity. If the cells are suspended in lower volumes, the cells are prone to aggregation. Administration of aggregated MSCs to animals has resulted in cardiac infarction. Thus, it is crucial that non-aggregated MSCs be administered according to the methods of the invention. The presence of HSA is also critical because it prevents aggregation of the MSCs and also prevents the cells from sticking to plastic containers the cells pass through when administered to subjects.

In certain embodiments of the method of producing MSCs of the invention, the culture system is used in conjunction with a medium for expansion of MSCs which does not contain any animal proteins, e.g. PL. FBS has been connected with adverse effects after in vivo application of FBS-expanded cells, e.g. formation of anti-FBS antibodies, anaphylactic or Arthus-like immune reactions or arrhythmias after cellular cardioplasty. FBS may introduce unwanted animal xenogeneic antigens, viral, prion and zoonose contaminations into cell preparations making new alternatives desirable.

Manufacturing Summary

In one embodiment, a bone marrow aspirate is suspended in culture media and then plated in multilayer cell factory. Mesenchymal progenitors naturally attach to the surface of the cell factory and then expand after several passages to become a relatively homogeneous population of MSC. After 1 to 3 days the cells remaining in suspension are washed out of the cell factory and discarded.

When the MSCs have expanded to cover the culture surface, they are enzymatically detached and harvested. The harvested cells are seeded in more cell factories and the expansion process is repeated. Feeding and harvesting of the cells takes place in a completely closed system using sterile welders.

After 2-6 rounds of expansion (12-20 days), the cells are harvested and cryopreserved in vapor phase liquid N₂ at <−130° C. Representative units are tested for sterility, mycoplasma, endotoxin, identity by flow cytometry and trilineage differentiation, as well as an array of viral tests.

Preferably, bone marrow aspirates are donated by healthy adult volunteers. Potential donors undergo rigorous testing including health questionnaire, physical examination, and testing for various infectious diseases.

Cryopreserved units (1-2) are thawed, cultured and expanded in a manner similar to the bone marrow aspirate cultures. The cells are expanded for two additional rounds at large scale to obtain the final product. The final harvested product is concentrated and washed using a scalable downstream process (e.g., Tangential Flow Filtration (TFF) and/or closed system centrifugation).

The MSC population is then packaged into cryogenic vials, frozen to −80° C. in a stepwise manner using a controlled rate freezer, and stored at <−130° C. in vapor phase liquid N₂. Moreover, the population is also tested for sterility, mycoplasma, endotoxin, and identity.

Unlike dead end filtration, TFF or closed system centrifugation is an efficient process for retaining and concentrating larger particulates (cells) while removing non-particulates (culture media). In TFF or closed system centrifugation, the system efficiently separates cells from culture media without the clogging that occurs in dead end filtration.

Determination of suitable protocols for cryopreservation and/or thawing of the MSCs prior to use are within the routine skill in the art.

Thus, this manufacturing system represents the next generation in cutting edge processes for MSC production. Specifically, it is scalable, performed in a closed culturing system, and free of animal origin products. Moreover, it employs a closed system centrifugation or TFF downstream processing system, which preserves cell viability. Likewise, it also uses a closed vialing system.

Methods of Using Mesenchymal Stem Cells

The MSCs can be used to treat or ameliorate conditions including, but not limited to, stroke, multi-organ failure (MOF), AKI of native kidneys, AKI of native kidneys in multi-organ failure, AKI in transplanted kidneys, kidney dysfunction, multi-organ dysfunction and wound repair that refer to conditions known to one of skill in the art. Descriptions of these conditions may be found in medical texts, such as Brenner & Rector's The Kidney, WB Saunders Co., Philadelphia, last edition, 2012, which is incorporated herein in its entirety by reference.

AKI is defined as an acute deterioration in kidney function within hours or days. In severe AKI, the urine output may be absent or very low. As a consequence of this abrupt loss in function, azotemia develops, defined as a rise of SCr and BUN levels. SCr and BUN levels are measured routinely or repeatedly in patients at risk for or following established AKI. When BUN levels have increased to approximately 10-fold their normal concentration, this corresponds with the development of uremic manifestations due to the parallel accumulation of uremic toxins in the blood. The accumulation of uremic toxins can cause bleeding from the intestines, neurological manifestations, most seriously affecting the brain, leading, unless treated, to coma, seizures and death. A normal SCr level is about 1.0 mg/dL, and a normal BUN level is about 20 mg/dL. In addition, acid (hydrogen ions) and potassium levels may rise rapidly and dangerously, resulting in cardiac arrhythmias and possible cardiac arrest and death. If fluid intake continues in the absence of urine output, the patient may become fluid overloaded, often resulting in a congested circulation, pulmonary edema and low blood oxygenation, thereby also threatening the patient's survival. One skilled in the art interprets these physical and laboratory abnormalities, and considers the prescription therapy based on the available information.

A decline in kidney function may be indicative of AKI in a subject. A decline in kidney function can be measured by an increase in one or more serum, blood, and/or urine biomarkers selected from serum creatinine (SCr), blood urea nitrogen (BUN), Cystatin C, Beta-trace protein (BTP) (also known as Prostaglandin D Synthase), Podocalyxin, Nephrin, Alpha 1-microglobulin, Beta 2-microglobulin, Glutathione S-transferase, Interleukin-18, Kidney Injury Molecule-1 (KIM-1), Liver-Type Fatty Acid-Binding Protein, Netrin-1, Neutrophil Gelatinase-Associated Lipocalcin (NGAL), and/or N-Acetyl-Beta-D-Glucosaminidase (NAG). A decline in kidney function can also be measured by an increase in serum creatinine (SCr) alone or in combination with an increase in one or more biomarkers selected from blood urea nitrogen (BUN), Cystatin C, Beta-trace protein (BTP) (also known as Prostaglandin D Synthase), Podocalyxin, Nephrin, Alpha 1-microglobulin, Beta 2-microglobulin, Glutathione S-transferase, Interleukin-18, Kidney Injury Molecule-1 (KIM-1), Liver-Type Fatty Acid-Binding Protein, Netrin-1, Neutrophil Gelatinase-Associated Lipocalcin (NGAL), and/or N-Acetyl-Beta-D-Glucosaminidase (NAG). The decline in kidney function can also be measured by an increase in one or more serum/blood biomarkers (e.g. SCr, BUN, Cystatin C, and/or BTP (also known as Prostaglandin D Synthase)) and/or an increase in one or more urine biomarkers (e.g., Podocalyxin, Nephrin, Alpha 1-microglobulin, Beta 2-microglobulin, Glutathione S-transferase, Interleukin-18, KIM-1, Liver-Type Fatty Acid-Binding Protein, Netrin-1, NGAL, and/or NAG).

By way of non-limiting example, the decline in kidney function can be measured by an increase in SCr levels of at least 0.3 mg/dL (e.g., 0.4 mg/dL, 0.5 mg/dL, or more).

Major causes of intrinsic AKI may include, for example:
tubular injury (e.g., ischemia due to hypoperfusion (i.e., hypovolemia, sepsis, hemorrhage, cirrhosis, congestive heart failure), endogenous toxins (i.e., myoglobin, hemoglobin, paraproteinemia, uric acid), and/or exogenous toxins (i.e., antibiotics, chemotherapy agents, radiocontrast agents, phosphate preparations));
tubulointerstitial injury (e.g., acute allergic interstitial nephritis (i.e., nonsteroidal anti-inflammatory drugs, antibiotics), infections (i.e., viral, bacterial, fungal infections), infiltration (i.e., lymphoma, leukemia, sarcoid), and/or allograft rejection));
glomerular injury (e.g., inflammation (i.e., anti-glomerular basement membrane disease, antineutrophil cytoplasmic autoantibody disease, infection, cryoglobulinemia, membraneoproliferative glomerulonephritis, Immunoglobulin A nephropathy, systemic lupus erythematosus) and/or hematologic disorders (i.e., Henoch-Schönlein purpuria, polyarteritis nodosa Hemolytic uremic syndrome, thrombotic thrombocytopenic purpura, drugs));
renal microvasculature (i.e., malignant hypertension, toxemia of pregnancy, hypercalcemia, radiocontrast agents, scleroderma, drugs); and/or
large vessels (e.g., arteries (i.e., thrombosis, vasculitis, dissection, thromboembolism, atheroembolism, trauma) and veins (i.e., thrombosis, compression, trauma)).

Moreover, causes of prerenal AKI may include, for example:
intravascular volume depletion (e.g., hemorrhage (i.e., trauma, surgery, postpartum, gastrointestinal), gastrointestinal losses (i.e., diarrhea, vomiting, nasogastric tube loss), renal losses (i.e., diuretic use, osmotic dieresis, diabetes insipidus), skin and mucous membrane losses (i.e., burns, hyperthermia), nephrotic syndrome, cirrhosis, or capillary leak); reduced cardiac output (e.g., cardiogenic shock, pericardial diseases (i.e., restrictive, constrictive, tamponade), congestive heart failure, valvular diseases, pulmonary diseases (i.e., pulmonary hypertension, pulmonary embolism), and/or sepsis);
systemic vasodilation (e.g., sepsis, cirrhosis, anaphylaxis, drugs);
renal vasoconstriction (e.g., early sepsis, hepatorenal syndrome, acute hypercalcemia, drugs (i.e., norepinephrine, vasopressin, nonsteroidal anti-inflammatory drugs, angiotension-converting enzyme inhibitors, calcineurin inhibitors), iodinated contrast agents); and/or
increased intraabdominal pressure (e.g., abdominal compartment syndrome).

Post renal causes of AKI may include, for example:
upper urinary tract extrinsic causes (e.g., retroperitoneal space (i.e., lymph nodes, tumors), pelvic or intraabdominal tumors (i.e., cervix, uterus, ovary, prostate), fibrosis (i.e., radiation, drugs, inflammatory conditions), ureteral ligation or surgical trauma, granulomatosis diseases, hematoma);
lower urinary tract causes (e.g., prostate (i.e., benign prostatic hypertrophy, carcinoma, infection), bladder (i.e., neck obstruction, calculi, carcinoma, infection (schistosomiasis)), functional (i.e., neurogenic bladder secondary to spinal cord injury, diabetes, multiple sclerosis, stroke, pharmacologic side effects of drugs (anticholinergics, antidepressants)), urethral (i.e., posterior urethral valves, strictures, trauma, infections, tuberculosis, tumors));
upper urinary tract intrinsic causes (e.g., nephrolithiasis, strictures, edema, debris (i.e., blood clots, sloughed papillae, fungal ball), malignancy).

A decrease in kidney function can be measured by an increase in SCr level of at least 0.3 mg/dL. This increase in SCr level (or level of other biomarker) is measured relative to a baseline level. For example, a baseline level of a biomarker (e.g., SCr level) can be a normal level measured in a control sample (i.e., in a subject or patient not suffering from or at risk of suffering form or developing kidney injury such as AKI) (e.g., a SCr level of about 1 mg/dL). Additionally or alternatively, a baseline level of a biomarker (e.g., SCr level) can be the level measured in the subject or patient suffering from, at risk of suffering from, or suspected of suffering from a kidney injury such as AKI at an earlier (e.g., at least 1 h, 2 h, 4 h, 8 h, 16 h, 32 h, 48 h, 3 d, 4 d, or 5 d) time point. A baseline level of a biomarker (e.g., SCr level) can also be determined from the subject or patient prior to a hospitalization and/or during hospitalization.

AKI can occur in clinical settings in a variety of patients, including, for example, AKI in cancer patients, AKI after cardiac surgery (e.g., after coronary artery bypass grafting, valve surgery, and/or other surgery utilizing cardiopulmonary bypass), AKI in pregnancy, AKI after solid organ or bone marrow transplantation, AKI and pulmonary disease (pulmonary-renal syndrome), AKI and liver disease, and AKI and nephrotic syndrome. (See Brenner and Rector's, The Kidney, WB Saunders Co., Philadelphia, 9th Edition (2012) (incorporated herein by reference in its entirety).

In addition, those skilled in the art will recognize that endogenous and/or exogenous toxins can cause acute tubular injury.

By way of non-limiting example, endogenous toxins may include, for example, myoglobulinuria; muscle breakdown (e.g., due to trauma, compression, electric shock, hypothermia, hyperthermia, seizures, exercise, burns, etc); metabolic disorders (e.g., hypokalemia, hypophosphatemia); infections (e.g., tetanus, influenza); toxins (e.g., isopropyl alcohol, ethanol, ethylene glycol, toluene, snake and insect bites, cocaine, heroin); drugs (e.g., hydroxymethylglutaryl-coenzyme A reductase inhibitors, amphetamines, fibrates); inherited diseases (e.g., deficiency of myophosphorylase, phosphofructokinase, carnitine palmityltransferase); autoimmune disorders (e.g., polymyositis, dermatomyositis); hemoglobinuria; mechanical causes (e.g., prosthetic valves, microangiopathic hemolytic anemia, extracorporeal circulation); drugs (e.g., hydralazine, methyldopa); chemicals (e.g., benzene, arsine, fava beans, glycerol, phenol); immunologic disorders (e.g., transfusion reaction); genetic disorders (e.g., glucose-6-phosphate dehydrogenase deficiency, paroxysomal nocturnal hemoglobinuria); hyperuricemia with hyperuricosuria; tumor lysis syndrome; hypoxanthane-guanine phosphoribosyltransferase deficiency; myeloma (e.g., light-chain production); and/or oxalate crystalluria (ethylene glycol).

Likewise, non-limiting examples of exogenous toxins can include, for example, antibiotics; aminoglycosides; amphotericin B; antiviral agents (e.g., acyclovir, cidofovir, indinavir, foscarnet, tenofovir); pentamidine; chemotherapeutic agents; ifosfamide; cisplatin; plicamycin; 5-Fluorouracil; cytarabine; 6-Thioguanine; calcineurin inhibitors; cyclosporin; tacrolimus; organic solvents; toluene; ethylene glycol; poisons; snake venom; paraquat; miscellaneous; radiocontrast agents; intravenous immune globulin; nonsteroidal antiinflammatory drugs; and/or oral phosphate bowel preparations.

Moreover, as shown below, various common drugs can be classified based in pathophysiologic categories of AKI:

| Pathophysiologic | |
|---|---|
| Category | Drugs |
| Vasoconstriction/ | Nonsteroidal antiinflammatory drugs |
| Impaired | (NSAIDs), angiotensin converting enzyme |
| Microvasculature | inhibitors, angiotensin receptor blockers, |
| Hemodynamics | norepinephrine, tacrolimus, cyclosporine, |
| (prerenal) | diuretics, cocaine, mitomycin C, estrogen, |
| | quinine, interleukin-2, cyclooxygenase-2 |
| | inhibitors |
| Tubular Cell Toxicity | Antibiotics (e.g., aminoglycosides, |
| | amphotericin B, vancomycin, rifampicin, |
| | foscarnet, pentamidine, cephaloridine, |
| | cephalothin), radiocontrast agents, NSAIDs, |
| | acetaminophen, cyclosporine, cisplatin, |
| | mannitol, heavy metals, intravenous immune |
| | globulin (IVIG), ifosfamide, tenofovir |
| Acute Interstitial | Antibiotics (e.g., ampicillin, penicillin G, |
| Nephritis | methicillin, oxacillin, rifampin in, |
| | ciprofloxacin, cephalothin, sulfonamides), |
| | NSAIDs, aspirin, fenoprofen, naproxen, |
| | piroxicam, phenylbutazone, radiocontrast |
| | agents, thiazide diuretics, phenytoin, |
| | furosemide, allopurinol, cimetidine, |
| | omeprazole |
| Tubular Lumen | Sulfonamides, acyclovir, cidofovir, |
| Obstruction | methotrexate, triamterene, methoxyflurane, |
| | protease inhibitors, ethylene glycol, indinavir, |
| | oral sodium phosphate bowel preparations |
| Thrombotic | Clopidogrel, cocaine, ticlopidine, cyclosporine, |
| Microangiopathy | tacrolimus, mitomycin C, oral contraceptives, |
| | gemcitabine, bevacizumab |
| Osmotic Nephrosis | IVIG, mannitol, dextrans, heat starch |

Multi-organ Failure (MOF) is a condition in which kidneys, lungs, liver and/or heart are impaired simultaneously or successively, associated with mortality rates as high as 100% despite the modern medical support. MOF patients frequently require intubation and respirator support because their lungs may develop Adult Respiratory Distress Syndrome (ARDS), resulting in inadequate oxygen uptake and CO₂ elimination. MOF patients may also depend on hemodynamic support, vasopressor drugs to maintain adequate blood pressures. MOF patients with liver failure may exhibit bleeding along with accumulation of toxins that often impair mental function. Patients may need blood transfusions and clotting factors to prevent or stop bleeding. It is considered that MOF patients may be given MSC therapy to address AKI and MOF.

Early graft dysfunction (EGD), delayed graft function (DGF), or transplant associated-acute kidney injury (TA-AKI) is AKI that affects the transplanted kidney in the first few days after implantation. The more severe TA-AKI, the more likely it is that patients will suffer from the same complications as those who have AKI in their native kidneys, as above. The severity of TA-AKI is also a determinant of enhanced graft loss due to rejection(s) in the subsequent years. These are two strong indications for the prompt treatment of TA-AKI with the MSCs of the present invention.

Chronic renal failure (CRF) or Chronic Kidney Disease (CKD) is the progressive loss of nephrons and consequent loss of renal function due to a variety of causes, including diabetic nephropathy and hypertensive nephropathy, resulting in End Stage Renal Disease (ESRD), at which time patient survival depends on dialysis support or kidney transplantation. The need for MSC therapy of the present invention will be determined on the basis of physical and laboratory abnormalities described above.

In some embodiments, the MSCs may be administered to patients in need thereof when one of skill in the art determines that conventional therapy fails. Conventional therapy includes hemodialysis, antimicrobial therapies, blood pressure medication, blood transfusions, intravenous nutrition and in some cases, ventilation on a respirator in the ICU. Hemodialysis is used to remove uremic toxins, improve azotemia, correct high acid and potassium levels, and eliminate excess fluid. In other embodiments of methods of use of MSCs of the invention, the MSCs of the invention are administered as a first line therapy. The methods of use of MSCs of the present invention is not limited to treatment once conventional therapy fails and may also be given immediately upon developing an injury or together with conventional therapy.

In certain embodiments, the MSCs are administered to a subject once. This one dose is sufficient treatment in some embodiments. In other embodiments the MSCs are administered 2, 3, 4, 5, 6, 7, 8, 9, 10, or more times in order to attain or sustain a therapeutic effect. For example, in some instances, the cells are administered chronically and/or on an on-going basis.

Monitoring patients for a therapeutic effect of the MSCs delivered to a patient in need thereof and assessing further treatment will be accomplished by techniques known to one of skill in the art. For example, renal function will be monitored by determination of SCr and BUN levels, serum electrolytes, measurement of renal blood flow (ultrasonic method), creatinine and insulin clearances, urine output, and other methods. A positive response to therapy for AKI includes return of excretory kidney function, normalization of urine output, blood chemistries and electrolytes, repair of the organ and survival. For MOF, positive responses also include improvement in blood pressure, blood oxygenation, and improvement in function of one or all organs.

In other embodiments the MSCs are used to effectively repopulate dead or dysfunctional kidney cells in subjects that are suffering from chronic kidney pathology including CKD. The effect may be the results of the paracrine and/or endocrine effects of the MSCs that induce endogenous progenitor cells in the kidney. Additionally (or alternatively), this effect may be because of the “plasticity” of the MSC populations. The term “plasticity” refers to the phenotypically broad differentiation potential of cells that originate from a defined stem cell population. MSC plasticity can include differentiation of stem cells derived from one organ into cell types of another organ. “Transdifferentiation” refers to the ability of a fully differentiated cell, derived from one germinal cell layer, to differentiate into a cell type that is derived from another germinal cell layer.

It was previously assumed that stem cells gradually lose their pluripotency and thus their differentiation potential during organogenesis. It was thought that the differentiation potential of somatic cells was restricted to cell types of the organ from which respective stem cells originate. This differentiation process was thought to be unidirectional and irreversible. However, recent studies have shown that somatic stem cells maintain some of their differentiation potential. (See Hombach-Klonich eta l., J Mol Med (Berl).86(12):1301-1314 (2008)). For example, stem cells may be able to transdifferentiate into muscle, neurons, liver, myocardial cells, and kidney cell populations. It is possible that as yet undefined signals that originate from injured and not from intact tissue act as transdifferentiation signals.

In certain embodiments, a therapeutically effective dose of MSCs is delivered to the patient. An effective dose for treatment will be determined by the body weight of the patient receiving treatment, and may be further modified, for example, based on the severity or phase of the stroke, kidney or other organ dysfunction, for example the severity of AKI, the phase of AKI in which therapy is initiated, and the simultaneous presence or absence of MOF. In some embodiments of the methods of use of the MSCs of the invention, from about 1×10⁵ to about 1×10¹⁰ MSCs per kilogram of recipient body weight are administered in a therapeutic dose. Preferably from about 1×10⁵ to about 1×10⁸ MSCs per kilogram of recipient body weight is administered in a therapeutic dose. More preferably from about 7×10⁵ to about 5×10⁸ MSCs per kilogram of recipient body weight is administered in a therapeutic dose. More preferably from about 1×10⁶ to about 1×10⁸ MSCs per kilogram of recipient body weight is administered in a therapeutic dose. More preferably from about 7×10⁵ to about 7×10⁶ MSCs per kilogram of recipient body weight is administered in a therapeutic dose. More preferably about 2×10⁶ to about 5×10⁶ MSCs per kilogram of recipient body weight is administered in a therapeutic dose. The number of MSCs used will depend on the weight and condition of the recipient, the number of or frequency of administrations, the route of administration, and other variables known to those of skill in the art. For example, a therapeutic dose may be one or more administrations of the therapy.

The therapeutic dose of MSCs is administered in a suitable solution for injection (i.e., infusion or bolus). Solutions are those that are biologically and physiologically compatible with the cells and with the recipient, such as buffered saline solution, Plasma-lyte or other suitable excipients or formulations, known to one of skill in the art.

In certain embodiments of the MSCs of the invention are administered to a subject at a rate between approximately 0.5 and 1.5 mL (e.g., 0.5, 0.6, 0.7, 0.8, 0.9, 1.0, 1.1, 1.2, 1.3, 1.4, or 1.5 mL) of MSCs in physiologically compatible solution per second. Preferably, the MSCs of the invention are administered to a subject at a rate between approximately 0.83 and 1.0 mL per second (e.g., 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0 mL). More preferably, the MSCs are suspended in approximately 100 mL of physiologically compatible solution and are completely injected into a subject between approximately one and three minutes. More preferably the 100 mL of MSCs in physiologically compatible solution is completely infused in approximately one to three minutes. Determination of injection and/or infusion rate for a given mode of administration is within the routine level of skill in the art.

In other embodiments, the MSCs are used in trauma or surgical patients scheduled to undergo high-risk surgery such as the repair of an aortic aneurysm. In the case of poor outcome, including infected and non-healing wounds, development of MOF post surgery, the patient's own MSCs, prepared according to the methods of the invention, that are cryopreserved may be thawed out and administered as detailed above. Patients with severe AKI affecting a transplanted kidney may either be treated with MSCs, prepared according to the methods of the invention, from an unrelated donor or the donor of the transplanted kidney (allogeneic) or with cells from the recipient (autologous). Allogeneic or autologous MSCs, prepared according to the methods of the invention, are an immediate treatment option in patients with TA-AKI and for the same reasons as described in patients with AKI of their native kidneys.

In certain embodiments, the MSCs of the invention are administered to the patient by infusion intravenously or intra-arterially (for example, for renal indications, via femoral artery into the supra-renal aorta). Preferably, the MSCs of the invention are administered via the supra-renal aorta. In certain embodiments, the MSCs of the invention are administered through a catheter that is inserted into the femoral artery at the groin. Preferably, the catheter has the same diameter as a 12-18 gauge needle. More preferably, the catheter has the same diameter as a 15 gauge needle. The diameter is relatively small to minimize damage to the skin and blood vessels of the subject during MSC administration. Preferably, the MSCs of the invention are administered at a pressure that is approximately 50% greater than the pressure in the subject's aorta. More preferably, the MSCs of the invention are administered at a pressure of between about 120 and 160 psi (e.g., about 120, 130, 140, 150, or 160 psi). Generally, at least 95% of the MSCs of the invention survive injection and/or infusion into the subject. Moreover, the MSCs are generally suspended in a physiologically acceptable carrier containing about 5-10% (e.g., 5, 6, 7, 8, 9, or 10%) HSA. The HSA, along with the concentration of the cells prevents the MSCs from sticking to the catheter or the syringe, which also insures a high (i.e. greater than 95%) rate of survival of the MSCs when they are administered to a subject. The catheter is advanced into the supra-renal aorta to a point approximately 20 cm above the renal arteries. Preferably, blood is aspirated to verify the intravascular placement and to flush the catheter. More preferably, the position of the catheter is confirmed through a radiographic or ultrasound based method. Preferably the methods are transesophageal echocardiography (TEE), an X-ray, or fluoroscopy. The MSCs of the invention are then transferred to a syringe that is connected to the femoral catheter. The MSCs, suspended in the physiologically compatible solution are then infused over approximately one to three minutes into the patient. Preferably, after injection and/or infusion of the MSCs of the invention, the femoral catheter is flushed with normal saline. Optionally, the pulse of the subject found in the feet is monitored, before, during and after administration of the MSCs of the invention. The pulse can be monitored to ensure that the MSCs do not clump during administration.

In certain embodiments, a therapeutically effective dose of MSCs is delivered intravenously (IV) to the patient. The therapeutic dose of MSCs in a suitable solution for injection is administered via IV injection, infusion, or bolus or other suitable methods into a peripheral, femoral, jugular, or other vein known to one of ordinary skill in the art.

Dose Rationale

A dose of 2×10⁶ human MSCs (hMSC)/kg of bodyweight of a preparation of human MSC designed for clinical use has been selected for further investigation of the preparation in clinical studies of AKI. Data from a Phase 1 study, other clinical investigations of hMSC, as well as nonclinical investigations support selection of this dose.

The Phase 1 study evaluated three dose levels of PL-produced hMSC, designated AC607, including 7×10⁵, 2×10⁶ and 7×10⁶ hMSC/kg. All doses of AC607 were safe and well tolerated in this study, with no treatment related adverse events or serious adverse events observed in any dose cohort. In other clinical studies, hMSC have been administered to subjects across a range of doses with no reported safety issues. Doses of hMSC in these other studies have typically ranged from 150 to 300 million MSC per subject (approximately 2 to 4×10⁶ MSC/kg for a 70-kg subject), consistent with the selected dose. (See Ankrum et al., Trends Mol. Med. 16(5):203-09 (2010)). Moreover, published data suggest that hMSC doses of at least 1×10⁶ MSC/kg are pharmacologically active in non-AKI clinical indications. (See Hare et al., J. Am. Coll Cardiol 2227-86 (2009)).

In a rat I/R model of AKI, hMSC at an intra-arterial dose of 1×10⁶ hMSC/kg significantly reduced serum creatinine (SCr) when administered to animals after the onset of AKI, as evidenced by a 7-fold increase in SCr. (See Cao et al., Biotechnol Lett 32:725-32 (2010)). Consistent with data for hMSC, a nonclinical study demonstrated that intra-arterial administration of rat MSC (rMSC) significantly lowered SCr in the rat I/R model of AKI at doses of 2×10⁶ rMSC/kg or 5×10⁶ rMSC/kg, but not at 0.5×10⁶ rMSC/kg. (See Tögel et al., Stem Cells Dev 18:475-85 (2009)). Further, another nonclinical investigation demonstrated that a single intra-arterial injection of rMSC at doses up to 15×10⁶ rMSC/kg was well tolerated in rats with AKI.

Collectively, these clinical and nonclinical data support selection of 2×10⁶ MSC/kg of AC607 as a safe and pharmacologically active dose for future clinical studies of AKI.

Clinical Data

In the Phase 1 study, a single intra-arterial injection of AC607 at 7×10⁵ hMSC/kg, 2×10⁶ hMSC/kg, or 7×10⁶ hMSC/kg was safe and well tolerated in 16 subjects undergoing elective cardiac surgery who were at risk for developing postoperative AKI.

In summary, a single, intra-arterial dose of up to 7×10⁶ hMSC/kg of AC607 was safe and well tolerated when administered to subjects after cardiac surgery.

Currently, there are over 150 clinical studies of hMSC (not limited to AKI trials) currently listed on ClinicalTrials.gov. In these clinical investigations, hMSC doses most commonly range from 2×10⁶ MSC/kg to 4×10⁶ MSC/kg. (See Ankrum et al., Trends Mol Med 16(5):203-209 (2010)). Moreover, hMSC have been safely administered to subjects at doses of up to 8×10⁶ MSC/kg with no reported treatment related adverse events. (See Kebriaei et la., Biol Blood Marrow Transplant. 15:804-11 (2009)).

In a double-blind, placebo-controlled study of 60 patients with acute myocardial infarction, subjects were randomized 2:1 to receive either hMSC or placebo. (See Hare et al., J Am Coll Cardiol 54:2227-86 (2009)). hMSC were administered at doses of 0.5×10⁶ MSC/kg, 1.6×10⁶ MSC/kg, or 5×10⁶ MSC/kg. The rate of arrhythmias was 4-fold less in subjects that received hMSC compared to the placebo group (8.8% versus 36.8%, P=0.025). hMSC-treated subjects experienced fewer premature ventricular contractions (PVC) compared to those treated with placebo (P=0.017), and the percentage of patients that experienced more than 10 PVC per hour was significantly reduced in hMSC-treated compared to placebo-treated subjects (10.0% versus 24%, P=0.001). Interestingly, the rate of PVC exhibited a dose-response effect with reductions in PVC detected in the 1.6×10⁶ MSC/kg and 5×10⁶ MSC/kg groups but not in the 0.5×10⁶ MSC/kg group, compared to the placebo group.

A randomized, multicenter, double-blind, placebo-controlled study is currently underway to test AC607 for the treatment of acute kidney injury in cardiac surgery subjects. See, ClinicalTrials.gov Identifier: NCT01602328, incorporated herein by reference. This phase 2 clinical study evaluates the safety and efficacy of AC607 for the treatment of kidney injury in cardiac surgery subjects (ACT-AKI). The clinical study will test about 200 subjects that are at least 21 years in age. Subjects entering the study will have undergone cardiac surgery, e.g., coronary artery bypass grafting, valve surgery, and/or other surgery utilizing cardiopulmonary bypass. Those who experience kidney injury within 48 hours of their surgery (e.g., subjects exhibiting laboratory evidence of kidney injury within 48 hours of surgery) will be enrolled in the study. For example, a subject enrolled in the study will have AKI, as measured by a 0.5 mg/dL or greater increase in SCr from baseline within 48 hours of surgery.

Once enrolled, subjects receive a single administration of AC607 or placebo (vehicle only). Subjects are randomly assigned (1:1 ratio) to AC607 or placebo, with approximately 100 subjects per group. In study, AC607 is provided as a single administration at a target dose of 2×10⁶ human MSC/kg body weight.

Safety and efficacy assessment are performed daily during the post-operative hospital stay from the day randomized into the study until discharge, at 30 days, and at 90 days after study drug administration (evaluation phase). In addition, safety and long-term clinical outcomes are assessed at 6, 12, 24, and 36 months after drug administration (long-term follow-up phase).

Kidney recovery is evaluated over the 30 days following AC607 administration. Death or the need for dialysis are evaluated within 90 days of dosing. After the 90 day evaluation period, subjects will enter a 3-year extension phase of the study to monitor safety and long-term outcomes (follow-up period). A primary outcome measure is time to kidney recovery. For example, time to kidney recovery is the time between administration of AC607 and the first occurrence of a post-dosing SCr level that is equal to or less than the subject's pre-operative baseline level. The pre-operative baseline SCr level is preferably measured within 30 days of surgery. If multiple laboratory results are available within the 30 days before surgery, the most recent SCr value prior to surgery is used to establish baseline. A secondary outcome measure is all-cause mortality or dialysis, for example, a subject who dies or receives dialysis within 30 and 90 days after dosing.

The MSCs (e.g., AC607) of the present invention can be administered to a subject in need thereof (e.g., a subject having undergone cardiac surgery). The type of cardiac surgery can include, but is not limited to, coronary artery bypass grafting, valve surgery, and/or any other surgery utilizing cardiopulmonary bypass. “Subjects in need thereof” can include subjects who experience kidney injury and/or a decline in kidney function within 6 days, 4 days, 48 hours, 24 hours, or 12 hours of cardiac surgery. Preferably, a subject in need thereof is one who experiences kidney injury and/or a decline in kidney function within 48 hours of cardiac surgery.

For example, in this study, a subject who experiences kidney injury and/or a decline in kidney function after cardiac surgery has an increase in serum creatinine level from baseline of at least 0.5 mg/dL. Alternatively or additionally, a subject who experiences kidney injury and/or a decline in kidney function after cardiac surgery has a SCr level greater than the normal SCr level (e.g., 1 mg/dL).

The MSCs described herein effectively treat and/or ameliorate AKI in subjects that have undergone cardiac surgery. The therapeutically effective dose of MSCs can be between about 7×10⁵ and about 7×10⁶ hMSC/kg bodyweight (e.g., about 7×10⁵, 8×10⁵, 9×10⁵, 1×10⁶, 2×10⁶, 3×10⁶, 4×10⁶, 5×10⁶, 6×10⁶, or 7×10⁶ hMSC/kg). Preferably, the therapeutically effective dose of MSCs is 2×10⁶ cells/kg bodyweight. The dose of MSCs can be provided to a subject in a single or multiple administrations (e.g., 2, 3, 4, 5, 6, 7, 8, 9, 10, or more administrations). Preferably, the dose of MSCs is provided in a single administration.

Therapeutic efficacy may be determined by any outcomes known in the art, including, but not limited to, time to kidney recovery, need for dialysis, death, and/or any other methods of assessment described herein. A time to kidney recovery that is reduced in subjects that have been administered hMSCs compared to subjects that have been administered placebo or no treatment indicates therapeutic efficacy of the hMSCs.

For example, the pre-operative baseline SCr level is determined from a subject within 30 days prior to surgery. Then, the SCr level is monitored after surgery and after dosing with hMSCs (e.g., within 30, 25, 20, 15, 10, 5, 2, or 1 days after dosing with hMSCs). The first occurrence of a post-dosing SCr level that is less than or equal to the pre-surgery SCr baseline level is the time to kidney recovery.

In another example, the post-dosing SCr level is compared to a normal SCr level (e.g., about 1.0 mg/dL). A post-dosing SCr level that is the same as or less than a normal SCr level indicates recovery from kidney injury and therapeutic effectiveness of the hMSCs.

In addition to SCr level, any other measurements of renal function described herein can also be used to evaluate therapeutic efficacy and time to kidney recovery. For example, a pre-operative baseline BUN level can be measured from a subject within 30 days (e.g., within 30, 25, 20, 15, 10, 5, 2, or 1 days) prior to surgery. Then, the BUN level is monitored after surgery and after dosing with hMSCs. The first occurrence of a post-dosing BUN level that is less than or equal to the pre-surgery BUN baseline level is the time to kidney recovery. In another example, the post-dosing BUN level is compared to a normal BUN level (e.g., about 20 mg/dL). A post-dosing BUN level that is the same as or less than a normal BUN level indicates recovery from kidney injury and therapeutic effectiveness of the hMSCs.

The need for kidney dialysis after cardiac surgery is also mitigated by administration of human MSCs. For example, the probability that a subject will require kidney dialysis (e.g., within 90 days, 60 days, 30 days, or less) after cardiac surgery is lower if treated with human MSCs than if treated with placebo or untreated. Additionally, the probability of death due to AKI after cardiac surgery (e.g., death after 30 days, 60 days, 90 days, 6 months, 12 months, 24 months, 36 months, or more) is lower in subjects treated with human MSCs of the present invention than untreated or treated with placebo.

The invention will be further illustrated in the following non-limiting examples.

### EXAMPLES

### Example 1

### Effect of Allogeneic MSC on AM in the Bilateral Renal I/R Rat Model

Primary endpoint: To determine if administration of allogeneic rat MSC (rMSC) at the time of reperfusion, or 24 or 48 hours post-reperfusion decreases the severity of renal injury in the bilateral renal I/R rat model of AKI compared to vehicle control, as measured by SCr concentration.

Secondary endpoints: To determine if administration of rMSC at the time of reperfusion, or 24 or 48 hours post-reperfusion decreases the severity of renal injury in the bilateral renal I/R rat model of AKI compared to vehicle control, as measured by BUN concentration or renal histopathology score.

rMSC were isolated from bone marrow taken from femurs and tibias of female Fischer 344 rats. Cells were passaged 5-6 times using culture medium (RMSC-GM) optimized for rMSC growth that contained 10% fetal bovine serum, 2 mM L-glutamine, and 1% gentamycin-amphotericin. The final rMSC product was cryopreserved in culture medium containing 10% DMSO and stored at ≦−132° C. in vapor phase liquid nitrogen. rMSC were positive for the cell surface markers CD29 and CD90, and negative for CD11b, CD34, and CD45 by flow cytometry. rMSC were capable of in vitro adipogenesis as indicated by Oil Red O staining, and osteogenesis as indicated by calcium mineralization. The final rMSC product was negative for Mycoplasma, bacteria, yeast and fungi and contained less than 0.5 EU/mL endotoxin.

AKI was induced in male Sprague-Dawley rats by bilateral renal I/R surgery (ischemia time=50 minutes). All animals were treated with either vehicle or allogeneic rMSC immediately after reperfusion (0 hours), and 24 and 48 hours post-reperfusion. Phosphate-buffered saline (PBS) vehicle or rMSC (5×10⁶ cells/kg) were administered intra-arterially via a carotid catheter that was implanted during the I/R surgery.

Male Sprague-Dawley rats were randomized based upon baseline SCr, BUN, and body weight, and divided into four groups (n=12/group):
Group A (vehicle) was treated with vehicle at 0, 24, and 48 hours post-reperfusion.
Group B (rMSC at 0 hours) was treated with rMSC at 0 hours after reperfusion, and with vehicle at 24 and 48 hours post-reperfusion.
Group C (rMSC at 24 hours) was treated with rMSC at 24 hours after reperfusion, and with vehicle at 0 and 48 hours post-reperfusion.
Group D (rMSC at 48 hours) was treated with rMSC at 48 hours after I/R surgery, and with vehicle at 0 and 24 hours post-reperfusion.

SCr and BUN were measured at baseline (prior to I/R), and at 24, 48, 72, 96, and 120 hours post-reperfusion. Animals were sacrificed at 120 hours, and both kidneys were collected for pathologic analysis.

Results

Live Phase

Treatment with rMSC at 0, 24, or 48 hours post-I/R abrogated AKI in the rat bilateral I/R AKI model (FIG. 1, Table 1). In the vehicle-treated group (A), SCr peaked at 24 hours post-I/R at 4.00 mg/dL and remained 4.4-fold higher at 120 hours post-I/R compared to baseline (0.93 and 0.21 mg/dL, respectively). SCr concentrations of animals treated with rMSC at 0 hours (Group B) were significantly decreased at all time points (i.e. 24, 48, 72, 96 and 120 hours post-I/R), compared to vehicle treated animals (P<0.05). SCr concentrations of animals treated with rMSC at 24 hours (Group C) were significantly decreased at 72 and 120 hours post-I/R compared to vehicle-treated animals (P<0.05) and were less than vehicle-treated animals at 48 hours (P=0.05). As expected, SCr concentrations of Group C animals did not differ from vehicle-treated animals at 24 hours post-I/R. Animals treated with rMSC at 48 hours (Group D) exhibited significantly lower SCr concentrations at all time points after MSC treatment (i.e., 72, 96 and 120 hours post-I/R) compared to vehicle-treated animals (P<0.05). As expected, SCr concentrations of Group D animals did not differ from vehicle-treated animals at 24 and 48 hours post-I/R.

TABLE 1
Summary of SCr Concentrations

| | Base- | 24 | 48 | 72 | 96 | 120 |
|---|---|---|---|---|---|---|
| | line | hours | hours | hours | hours | hours |
| Vehicle | 0.21 | 4.00 | 3.67 | 2.64 | 1.62 | 0.93 |
| only | | | | | | |
| rMSC at | 0.21 | 3.07* | 2.23* | 1.26** | 0.77** | 0.50** |
| 0 hours | | | | | | |
| rMSC at | 0.21 | 3.64 | 2.84 | 1.59* | 1.00^{¶} | 0.56* |
| 24 hours | | | | | | |
| rMSC at | 0.21 | 3.83 | 2.66 | 1.34* | 0.81* | 0.52* |
| 48 hours | | | | | | |

Mean SCr concentration (mg/dL) data are expressed as means. Two-way ANOVA analysis using JMP software was conducted to assess differences between means for each rMSC-treated group compared to the vehicle-treated group.
*P <0.05, **P <0.01, ^{¶}P = 0.05.

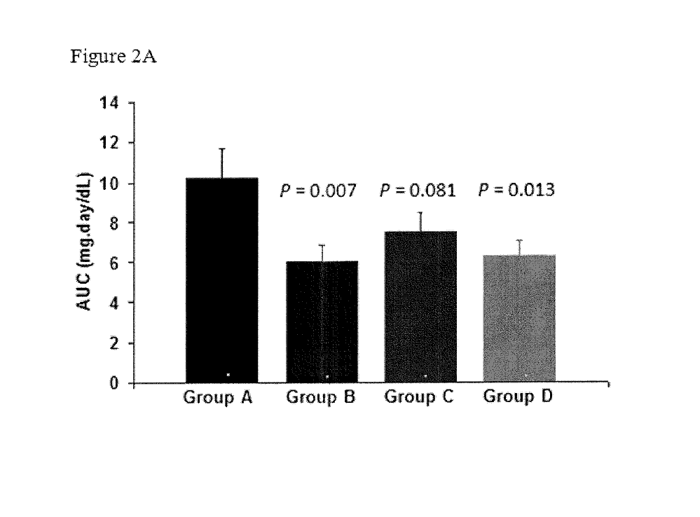

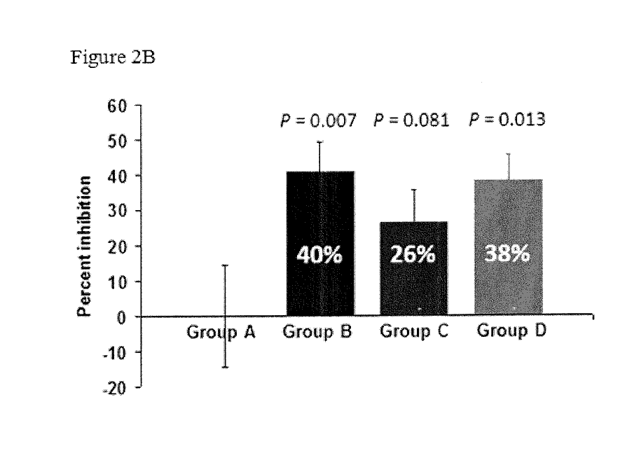

As shown in FIG. 2, rMSC administered at 0 hours post-I/R showed the most prominent abrogation of AKI with a 40% reduction in the SCr area under the curve (AUC) compared to vehicle-treated animals (P=0.007), followed by rMSC administered at 48 hours with 38% reduction in SCr AUC (P=0.013). The 26% reduction in SCr AUC observed in animals treated with rMSC at 24 hours was not statistically significant (P=0.081).

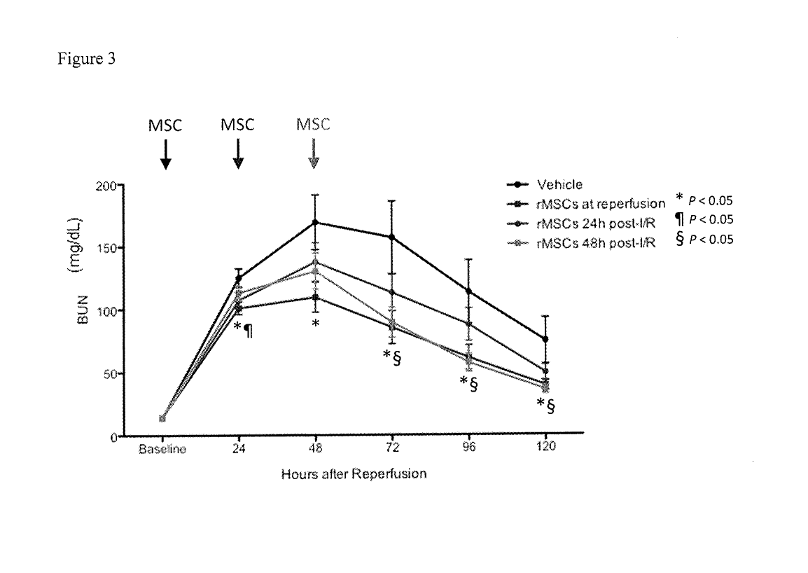

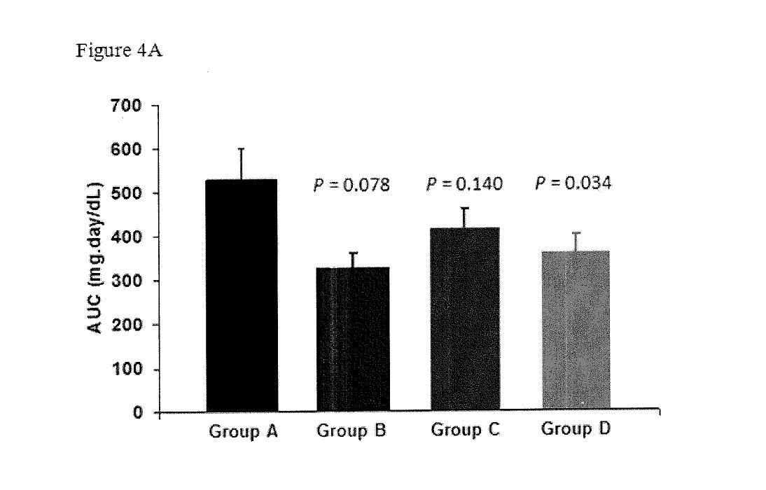

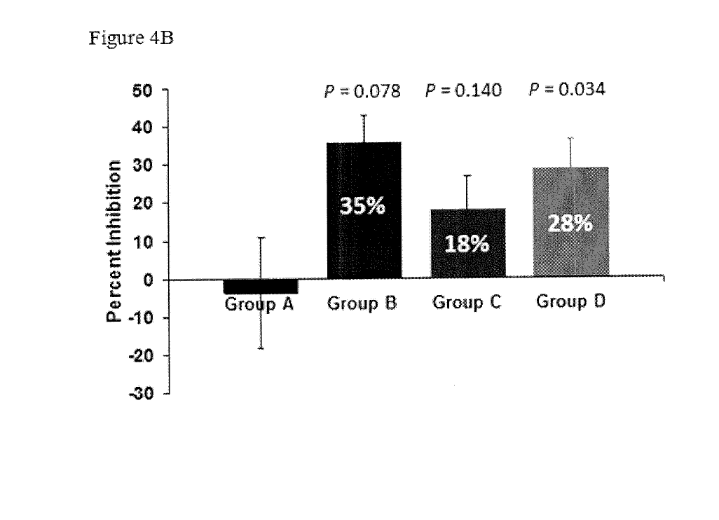

Serum BUN concentrations showed similar trends to those observed for SCr (FIG. 3, Table 2). Animals treated with rMSC at 0 hours (Group B) showed statistically lower BUN concentrations at all time points after rMSC treatment (i.e., 24, 48, 72, 96 and 120 hours post-I/R) compared to vehicle-treated animals (P<0.05). Animals treated with rMSC at 48 hours (Group D) exhibited significantly lower BUN concentrations at all time points after rMSC treatment (i.e., 72, 96 and 120 hours post-UR, compared to vehicle-treated animals (P<0.05). Group C showed similar trends in BUN concentrations as observed for other rMSC-treated groups, however, the differences were only significant at 24 hours post-I/R (P<0.05).

TABLE 2
Summary of BUN Concentrations

| | Base- | 24 | 48 | 72 | 96 | 120 |
|---|---|---|---|---|---|---|
| | line | hours | hours | hours | hours | hours |
| Vehicle | 13.78 | 125.03 | 169.22 | 156.93 | 113.46 | 74.61 |
| only | | | | | | |
| rMSC at | 14.11 | 101.09** | 109.63* | 85.46** | 61.34* | 39.21* |
| 0 hours | | | | | | |
| rMSC at | 14.00 | 107.30* | 137.61 | 113.09 | 87.50 | 49.35 |
| 24 hours | | | | | | |
| rMSC at | 13.95 | 113.10 | 130.27 | 89.41* | 57.08* | 35.73* |
| 48 hours | | | | | | |

Mean BUN concentration (mg/dL) data are expressed as means. Two-way ANOVA analysis using JMP software was conducted to assess differences between means for each rMSC-treated group compared to the vehicle-treated group.
*P <0.05, **P <0.01.

Similar trends in BUN AUC values, as for SCr AUC values, were observed. Specifically, compared to vehicle-treated animals, the BUN AUC was reduced by 35% in animals treated with rMSC at 0 hours (P=0.078), by 18% in animals treated with rMSC at 24 hours (P=0.14), and by 28% animals treated with rMSC at 48 hours (P=0.034).

During the course of the experiment, rMSC-treated animals did not experience greater losses of body weight compared to vehicle-treated animals (decreases of 6.7−8.8% versus 9.6% for rMSC-treated and vehicle-treated animals, respectively). rMSC-treated animals did not show any clinical signs or symptoms. Three animals died during the course of the experiment: one animal from Group A (vehicle) and two animals from Group D (rMSC at 48 hours), as detailed below in Pathology. One of the two deaths in Group D was due to termination of an animal after hemorrhage caused by displacement of the carotid catheter.

Pathology

Methods

The right and left kidneys were collected as per protocol. Hematoxylin and eosin (H&E)-stained slides were prepared from the collected kidneys and evaluated microscopically. Renal lesions were qualitatively graded using the grading scheme in Table 3.

Results

Morbidity (and subsequent euthanasia) or mortality occurred in individual rats in Groups A (Number 12) and D (Numbers 46 and 53). The cause of death in Rats 12 and 46 was considered acute and severe model-related kidney injury as evidenced by the characteristic lesions of this model (see below) and evidence of hemorrhage in the urine (when available). In one rat in Group D (Number 53), displacement of the carotid catheter at the site of externalization (ventral cervical region) and subsequent hemorrhage was considered the cause of death. Macroscopic and microscopic changes in the kidney of this rat were also considered consistent with the model.

Important model-related effects in rats in all groups in this study were similar to that described for this model (Vogt and Farber, 1968; Shanley et al, 1986) and consisted of macroscopic enlargement and/or pallor of the affected kidney(s) with variable prominence of a distinct white line at the corticomedullary junction on the cut surface. These macroscopic changes correlated with variable tubular epithelial degeneration/necrosis (Table 4) centered on the corticomedullary junction, consistent with vascular occlusion of the renal vessels and their tributaries, the arcuate vessels. Of note, model-related changes in rats in all groups were generally of greater severity in the right kidney, as compared to the left, likely relating to regional differences in the anatomic placement of the kidney in the right versus left side of the abdominal cavity combined with the surgical procedure of clamp application.

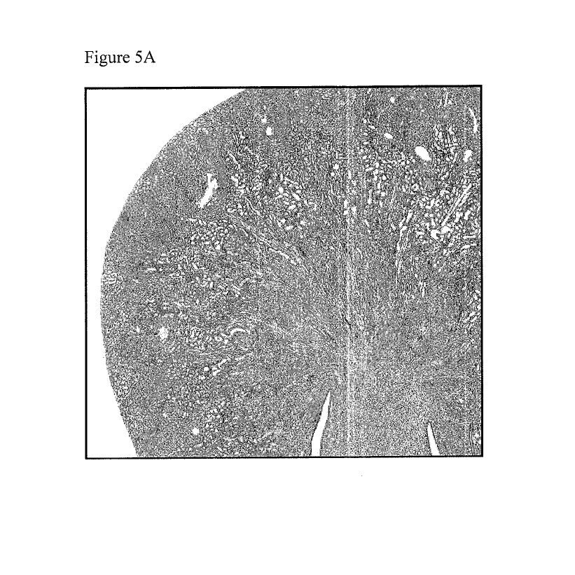

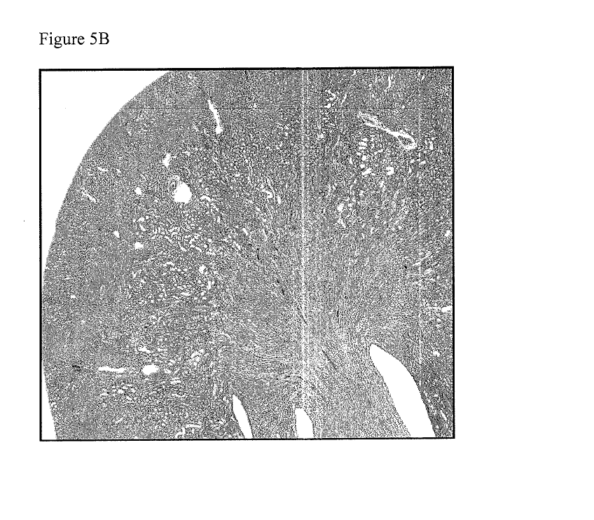

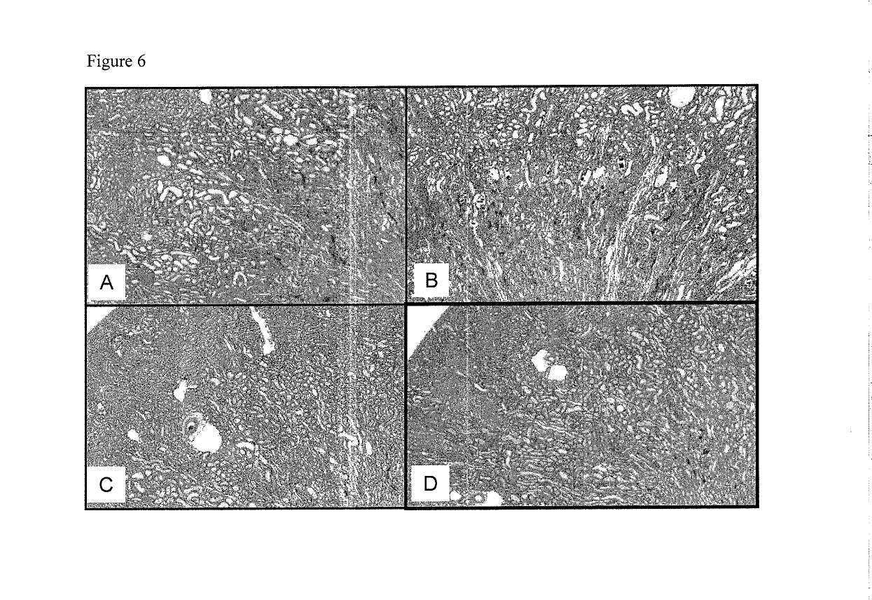

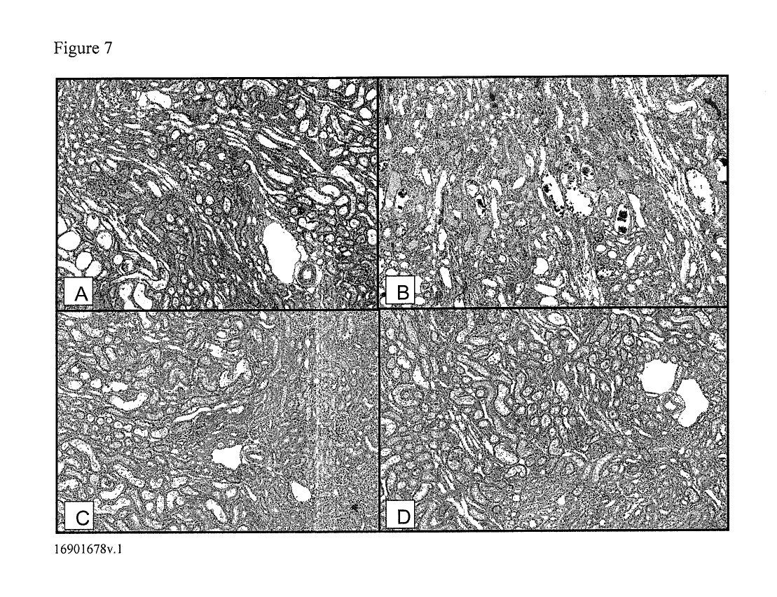

Tubular epithelial degeneration/necrosis was characterized by loss, fragmentation and/or attenuation of tubular epithelium (FIGS. 7A and B). The predominantly affected tubules were those of the medulla (outer stripe and medullary rays) and adjacent cortex, consistent with the occlusion and reperfusion of the vessels supplying these areas (FIGS. 5-7). In the more severely affected rats, this finding expanded into the cortex to a greater extent with a larger number of cortical tubules apparently affected (FIGS. 5A and 6A). Affected tubules were variably dilated (which contributed to the attenuation of the tubular epithelium) and contained exfoliated, degenerating/necrotic cells and cellular and/or granular eosinophilic debris (casts), occasionally admixed with proteinaceous fluid (FIG. 6A-B; 7A-B). In the more severely affected rats, tubular changes were accompanied by tubular mineralization (FIG. 7B) and the presence of proteinaceous fluid in tubules in the renal papilla as well as those in the medulla.

The interstitium in the affected parenchyma (medulla, cortex, and corticomedullary junction) occasionally contained individualized to accumulated fibroblasts, considered consistent with fibroplasia. In the majority of all rats in all treatment groups, these changes were accompanied by variable tubular regeneration, indicating an attempt at tissue repair (FIGS. 6-7). Characteristics of tubular regeneration consisted of increased cytoplasmic basophilia of the epithelial cells accompanied by anisocytosis, anisokaryosis, and variable nuclear:cytoplasmic (N:C) ratios within the tubular epithelium.

Multifocal, lymphocytic inflammation was present in one or both kidneys in individual rats in all groups and was considered consistent with a common spontaneous change in this age, gender, and strain of rat. In contrast, in individual rats in all groups, multifocal lymphohistiocytic and neutrophilic inflammation in one or both kidneys differed from the aforementioned background inflammation due to the presence of neutrophils and its generalized localization to the model-related injury. However, the neutrophilic and lymphohistiocytic inflammation was considered an individual manifestation of model-related effects rather than a compound-related effect due to the absence of a duration-dependent effect in the compound-treated groups and the overall generally low incidence of the finding.

Concurrent renal insufficiency/failure was indicated by increases in concentrations of SCr and BUN (Table 4 and detailed above in Results). The changes in these parameters in all groups were evident and generally of the greatest magnitude at 24 hours post-I/R, declined in magnitude beginning at 72 hours post-I/R and continuing until 120 hours post-I/R, where they remained increased but substantially diminished compared to earlier time points.

Importantly, the model-related injury observed pathologically and concurrent increases in SCr and BUN concentrations were of greatest magnitude and severity in vehicle-treated rats (Group A, Table 4). In contrast, administration of rMSC at 0, 24, or 48 hours post-I/R (Groups B, C, and D, respectively) reduced the severity of the ischemia/reperfusion injury observed in histopathology and also reduced the concurrent increases in SCr and BUN concentrations, compared to vehicle-treated animals (Group A).

The treatment-related (rMSC-mediated) effects in Groups B, C, and D are summarized below.

Group B (rMSC Administered at 0 Hours Post-Reperfusion)
The treatment-related attenuation of the model-related renal injury was most striking in rats in this group in which rMSC were given immediately after reperfusion at 0 hours after I/R surgery. Specifically, rats in this group exhibited a diminished magnitude of increase in the clinical pathology parameters (i.e., SCr and BUN), compared to that of vehicle-treated rats. Duration-dependent decreases in the indicators of renal injury (SCr and BUN concentrations) were evident at all time points after rMSC treatment until study termination (i.e., 24 hours through 120 hours post-I/R).
This apparent rMSC-mediated attenuation of model-related effects on the clinical pathology parameters was supported by the number of rats, at the time of study termination, with diminished severity of microscopic lesions in both kidneys in rMSC-treated rats (Group B; Table 4), as compared to the vehicle-treated controls (Group A; Table 4). Specifically, no Group B animals were found to have tubular epithelial degeneration/necrosis severity scores of severe, whereas 2 vehicle-treated rats (Group A) had severity scores of severe. Further, Group B animals also had lesser incidence of tubular epithelial degeneration/necrosis severity scores of marked compared to vehicle-treated-animals (i.e., incidence of rats with grade of marked, left kidney: 1 and 9 for Groups B and A, respectively; incidence of rats with grade of marked, right kidney: 3 and 8 for Groups B and A, respectively).

Group C and D (rMSCs Administered at 24 and 48 Hours Post-Reperfusion, Respectively)
Treatment-related attenuation of the model-related renal injury in these groups was evident in clinical pathology parameters (i.e., SCr and BUN concentration) at 72 hours post-reperfusion and exhibited a duration-dependent decrease in magnitude, compared to the vehicle-treated group until study termination (120 hours post-I/R).
This apparent rMSC-mediated attenuation of model-related effects on the clinical pathology parameters was supported by the number of rats with diminished severity of the lesions in both kidneys (Groups C and D; Table 4), as compared to the vehicle-treated controls (Group A; Table 4), as well as a lower number of rats with a more pronounced severity grade at the time of termination. The specifics of these results are described below.

No Group C animals and only 1 Group D animal were found to have tubular epithelial degeneration/necrosis severity scores of severe, compared to severe scores for 2 vehicle-treated rats (Group A).

Further, both Groups C and D animals also had lesser incidence of tubular epithelial degeneration/necrosis severity scores of marked as compared to vehicle-treated-animals (Group A). Specifically, rats in Group C had scores of marked in the left kidney in 1 versus 9 rats (vehicle-treated) and, in the right kidney, in 3 versus 8 rats (vehicle-treated). Similarly, rats in Group D had scores of marked in the left kidney in 0 versus 9 rats (vehicle-treated) and, in the right kidney, in 2 versus 8 rats (vehicle-treated).
Importantly, the apparent rMSC-mediated attenuation of the model-related renal injury, as indicated by the histological changes, was generally comparable between rats treated with rMSC at 24 (Table 4; Group C) or 48 (Table 4; Group D) hours post-I/R.

Indwelling carotid catheters placed in all rats were evaluated macroscopically at the time of necropsy and were found to terminate in the aorta, specifically the aortic arch.

TABLE 3
Grading Scheme for Renal Tubular Degeneration/Necrosis (Hematoxylin
and Eosin-Stained Slides) in Rats Given Vehicle or rMSC in the
Bilateral Renal I/R AKI Model

| Severity Grade | Description |
|---|---|
| Grade 0 (No visible | No changes or changes consistent with |
| lesions; NVL) | spontaneous background finding in the age, |
| | gender, and/or strain of rat. |
| Grade 1 (Minimal, MI) | Approximately 0-10% of the tubules are |
| | affected. |
| Grade 2 (Slight, SL) | Approximately >10% to 25% of the tubules are |
| | affected. |
| Grade 3 (Moderate, MO) | Approximately >25% to 50% of the tubules in |
| | the kidney section are affected. |
| Grade 4 (Marked, MA) | Approximately >50% to 75% of the tubules in |
| | the kidney section are affected. |
| Grade 5 (Severe, SE) | Approximately >75% of the tubules in the |
| | kidney section are affected. |

TABLE 4
Summary of Changes in Rats Given Vehicle or rMSC in
the Bilateral Renal I/R AKI Model

| | Group | | | |
|---|---|---|---|---|
| | A | B | C | D |
| Administration of | NA | 0 | 24 | 48 |
| rMSC (hours relative to | (vehicle- | | | |
| reperfusion)^{a} | treated) | | | |
| Number of | 12 M | 12 M | 12 M | 12 M |
| Animals/Group | | | | |
| Mortality | | | | |
| Found dead or | 1 | — | — | 2 |
| euthanized early | | | | |
| Clinical Chemistry | | | | |
| | Percent Change from Pretreatment, Severity | | | |
| | (24 hours post-reperfusion) | | | |
| BUN | ↑807 MA | ↑617 MA | ↑670 MA | ↑772 MA |
| Creatinine | ↑1777 SE | ↑1368 SE | ↑1704 SE | ↑1714 SE |
| | Percent Change from Pretreatment, Severity | | | |
| | (48 hours post-reperfusion) | | | |
| BUN | ↑1128 SE | ↑677 MA | ↑907 MA | ↑903 MA |
| Creatinine | ↑1621 SE | ↑968 MA | ↑1380 SE | ↑1269 SE |
| | Percent Change from Pretreatment, Severity | | | |
| | (72 hours post-reperfusion) | | | |
| BUN | ↑1039 SE | ↑506 MA | ↑771 MA | ↑684 MA |
| Creatinine | ↑1136 SE | ↑500 MA | ↑781 MA | ↑733 MA |
| | Percent Change from Pretreatment, Severity | | | |
| | (120 hours post-reperfusion) | | | |
| BUN | ↑441 MA | ↑178 SL | ↑336 MO | ↑357 MO |
| Creatinine | ↑336 MO | ↑137 SL | ↑239 MO | ↑303 MO |
| Anatomic Pathology | Number of Animals Affected, Severity | | | |
| Kidney (Left) | | | | |
| Tubular epithelial | | 1 MI | | |
| degeneration/necrosis | | 5 SL | 5 SL | 6 SL |
| | 3 MO | 5 MO | 6 MO | 6 MO |
| | 9 MA | 1 MA | 1 MA | |
| Inflammation, | 1 MI | 1 MI | | |
| lymphohisiocytic and | | 2 SL | | |
| neutrophilic | | | — | 1 MA |
| Kidney (Right) | | | | |
| Tubular epithelial | | 4 SL | 2 SL | 2 SL |
| degeneration/necrosis | 2 MO | 5 MO | 7 MO | 7 MO |
| | 8 MA | 3 MA | 3 MA | 2 MA |
| | 2 SE | | | 1 SE |
| Inflammation, | | 1 MI | | |
| lymphohisiocytic and | | 1SL | | |
| neutrophilic | — | 1 MO | 1 MO | |
| | | | 1 MA | 1 MA |

Abbreviations:
M = male;
= no important changes or finding not observed;
↑ = increased;
NA = Not applicable
Percent change from pretreatment values = [(mean treated value − mean pretreatment value)/mean pretreatment value] × 100
Severity grading scale: minimal (MI), slight (SL), moderate (MO), marked (MA), severe (SE).
^{a}Rats in groups B, C, and D received 5 × 10⁶ rMSC/kg

### Example 2

### DNA Isolation from Human Blood Samples

The objective of this Example is to ensure that a sufficient quantity of DNA is isolated from human blood samples using the Qiagen DNeasy Blood and Tissue Kit for subsequent determination of the GT repeat lengths in both HO-1 promoter alleles. This protocol is designed for use in the isolation of total DNA from human blood samples. DNA samples are sent to an outside vendor for fragment length analysis to determine the GT repeat lengths in the HO-1 promoter region.

Required Materials
1. Anti-coagulated human blood in and EDTA-vacutainer (from a refrigerated or a thawed, frozen sample)
2. Qiagen DNeasy Blood & Tissue Kit (Cat. #69504)
   Proteinase K
   Buffer AL
   Buffer AW 1
   Buffer AW2
   Buffer AE
   Spin Columns
   Collection Tubes
3. Ethanol (96-100%)
4. Water bath set to 56° C.
5. 1.5 mL microcentrifuge tubes
6. Phosphate-buffered saline (PS), Lonza catalog #17-513F (or equivalent)
7. Assorted serological pipettes

25 mL ethanol was added to Buffer AW and 30 mL ethanol was added to Buffer AW2 prior to procedure. All centrifugations were performed at room temperature. Four separate DNeasy columns were used for each donor's blood sample, and the 4 DNA samples purified from the same donor were combined at the end of the purification procedure.

Procedure
1. For each blood sample, 4 microcentrifuge tubes were with the blood sample identification.
2. 20 μl proteinase K were added to each of the 4 microcentrifuge tubes. The blood sample vacutainer tube was thoroughly mixed by vortexing and 100 μl anti-coagulated blood was transferred to each microcentrifuge tube, then 100 μl PBS was added to each microcentrifuge tube.
3. Vacutainer tube was capped and wrapped with parafilm. The remaining blood was stored in the freezer.
4. 200 μL Buffer AL was added to each microcentrifuge tube and mixed thoroughly by vortexing. Tubes were incubated at 56° C. for 10 minutes.
5. 200 μL ethanol (96-100%) were added to each tube and mixed thoroughly by vortexing.
6. The mixture was pipette from each tube into a separate DNeasy Mini spin column placed in a 2 mL collection tube. Tubes were centrifuged for 1 min at ≧6000×g. Flow-through and collection tube were discarded.
7. Each spin column was placed in a fresh 2 mL collection tube. 500 μl Buffer AW1 was added to each spin column. Tubes were centrifuged for 1 min at ≧6000×g. Flow-through and collection tube were discarded.
8. Each spin column was placed in a fresh 2 mL collection tube. 500 μl Buffer AW2 was added to each spin column. Tubes were centrifuged for 3 min at ≧20,000×g (14,000 rpm). Flow-through and collection tube were discarded.
9. Each spin column was transferred to a fresh 1.5 mL micro-centrifuge tube. DNA was eluted by adding 200 μl Buffer AE to the center of each spin column membrane. Tubes were incubated for 1 minute at room temperature (15-25° C.) and were centrifuged for 1 minute at ≧6000×g.
10. The 4 DNA samples purified from the same donor were combined into a single 1.5 L microcentrifuge tube.
11. The purified DNA was quantitated by measuring the optical density (OD) 260.
   a. 20 μl of the combined DNA sample was diluted with 80 μl of water in a fresh 1.5 mL tube.
b. the diluted DNA was pipette into a well of a 96-well UV compatible plate.
c. the OD at 260 and 280 nanometers was measured.
d. the formula of OD_{260/280} of 1=50 μg/mL DNA was used
   i. For example, an OD_{260/280} of 0.015=0.75 μg/mL DNA
   e. the DNA concentration was confirmed using the nanodrop method, if available.
12. DNA sample tube was stored at −20° C.
13. Date of DNA isolation was recorded.
14. A sufficient quantity of DNA was submitted for fragment analysis. The GT repeat length was determined by comparing the resulting fragment size to the published HO-1 promoter sequence and fragment sizes of synthetic DNA fragments with known GT repeat lengths.

### Example 3

### DNA Isolation from Cryopreserved MSC

The objective of this Example is to ensure that a sufficient quantity of DNA is isolated from cryopreserved MSC samples using the Qiagen DNeasy Blood and Tissue Kit for subsequent determination of the GT repeat lengths in both alleles of the HO-1 promoter. This protocol is designed for use in the isolation of total DNA from frozen MSC samples. DNA samples are sent to an outside vendor for fragment length analysis to determine the GT repeat lengths in the HO-1 promoter region.

Required Materials
1. Cryopreserved MSC
2. Qiagen DNeasy Blood & Tissue Kit (Cat. #69504)
   Proteinase K
   Buffer AL
   Buffer AW 1
   Buffer AW2
   Buffer AE
   Spin Columns
   Collection Tubes
3. Ethanol (96-100%)
4. Water bath set to 56° C.
5. 1.5 mL microcentrifuge tubes
6. Phosphate-buffered saline (PS), Lonza catalog #17-513F (or equivalent)
7. Assorted serological pipettes

25 mL ethanol was added to Buffer AW and 30 mL ethanol was added to Buffer AW2 prior to procedure. All centrifugations were performed at room temperature.

Procedure
1. A frozen MSC sample (approximately 1×10⁵ to 5×10⁶ MSC) was thawed in a 37° C. water bath and the cells were transferred to a 1.5 mL microcentrifuge tube. Cells were spun for 1 minute at 6000×g (8000 rpm). Supernatant was aspirated and 200 PBS was added, mixed, and then 20 μL Proteinase K was added.
2. 200 μL Buffer AL was added and mixed thoroughly by vortexing. Tubes were incubated at 56° C. for 10 minutes.
3. 200 μL ethanol (96-100%) was and mixed thoroughly by vortexing.
4. The mixture was pipetted into a DNeasy Mini spin column placed in a 2 mL collection tube and centrifuged for 1 min at ≧6000×g. Flow-through and collection tube were discarded.
5. The spin column was placed in a fresh 2 mL collection tube. 500 μl Buffer AW1 was added and tube was centrifuged for 1 min at ≧6000×g. Flow-through and collection tube were discarded.
6. Spin column was placed in a fresh 2 mL collection tube. 500 μl Buffer AW2 was added and tube was centrifuged for 3 min at ≧20,000×g (14,000 rpm). Flow-through and collection tube were discarded.
7. Spin column was transferred to a fresh 1.5 mL micro-centrifuge tube. DNA was eluted by adding 200 μl Buffer AE to the center of the spin column membrane and tube was incubated for 1 minute at room temperature (15-25° C.) and centrifuged for 1 minute at ≧6000×g.
8. DNA was quantitated by measuring the optical density (OD) 260.
   a. 20 μl of the DNA sample was diluted with 80 μl of water in a fresh 1.5 mL tube.
   b. the diluted DNA was pipette into a well of a 96-well UV compatible plate.
   c. the OD at 260 and 280 nanometers was measured.
   d. the formula of OD_{260/280} of 1=50 μg/mL DNA was used
      i. For example, an OD_{260/280} of 0.015=0.75 μg/mL DNA
   e. the DNA concentration was confirmed using the nanodrop method, if available.
9. DNA was stored at −20° C.
10. A sufficient quantity of DNA was submitted for fragment analysis. The GT repeat length was determined by comparing the resulting fragment size to the published HO-1 promoter sequence and fragment sizes synthetic DNA fragments with known GT repeat lengths.

### Example 4

### Human HO-1 Gene Promoter GT Repeat Analysis

The objective of this example is to determine the number of GT repeats in the human HO-1 gene promoter using fragment length analysis. Total DNA purified from human blood (see Example 1, supra) or MSC samples (see Example 2, supra) were submitted to an outside vendor (University of Utah Genetics Core Facility) for fragment length analysis. Polymerase chain reaction (PCR) using a specific, forward oligonucleotides primer labeled with 6-fluorescein amidite (6-FMA) and a specific, unlabeled reverse primer flanking the GT-repeats within the HO-1 promoter were used to synthesize 6-FAM labeled DNA fragments. Fragment length analysis of the 6-FAM labeled PCR products were conducted by the outside vendor to determine the number of GT repeats in the HO-1 promoter region.

Required Materials
1. Total DNA purified from blood or cells using DNeasy kit −50-100 ng per sample is needed.
2. Control DNA from Master Cell Bank (MCB) 808 or MCB 810 (50-100 ng per sample).
3. Reverse-phase HPLC purified 6-FAM labeled forward primer, synthesized and labeled by integrated technologies (IDT)

| | |
|---|---|
| forward primer sequence | |
| 5′-6-FAM-TGACATTTTAGGGAGCTGGAGACA | (SEQ ID NO: 1) |

the forward primer will be diluted to a 10 μM solution and used as 1 μL per 20 μL PCR reaction.
4. Reversed-phase HPLC purified unlabeled reverse primer

| | |
|---|---|
| reverse primer sequence | |
| 5′-ACAAAGTCTGGCCATAGGAC | (SEQ ID NO: 2) |

the reverse primer will be diluted to a 10 μM solution and used as 1 μL per 20 μL PCR reaction.
5. Microcentrifuge tubes (1.5 mL)

DNA purified from human blood or MSC samples using Qiagen's DNeasy blood and tissue kit #69504 were used. For positive controls, DNA from MCB 808 or other samples, such as synthetic DNA with known fragment lengths using the same PCR primers were submitted.

Procedure
1. 50-100 ng of total DNA from each sample to be genotyped (or positive control DNA) were aliquoted into separate 1.5 mL microcentrifuge tubes.
2. 50 μL of the 50 μM forward and reverse primer stock solutions were aliquoted into separate 1.5 mL microcentrifuge tubes. The primers were diluted to a 10 μM working solution and were used at 1 μL, PCR reactions at the external vendor.
3. The DNA samples and primer stock solutions were submitted to the external vendor.
4. Any remaining volume of the primers remained at the vendor for future PCR and fragment length analysis.

Data Analysis
1. Fragment length data received from external vendor.
2. Confirmed that the positive control (e.g., MCB 808 and 810) fragments were the expected length (in base pairs), as predicted from the published HO-1 promoter sequence.
3. Fragment sizes (in base pairs) were determined for submitted DNA samples from the plots received from the vendor.
4. Sizes of fragments and numbers of GT repeats for each sample were recorded.

### Example 5

### Preparation of PL

A MSC expansion medium containing PL was developed as an alternative to FBS. PL isolated from platelet rich plasma (PRP) were analyzed with either Human 27-plex (from BIO-RAD) or ELISA to show that inflammatory and anti-inflammatory cytokines as well as a variety of mitogenic factors are contained in PL, as shown below in Table 5. The human-plex method presented the concentration in [pg/mL] from undiluted PL while in the ELISA the PL was diluted to a thrombocyte concentration of 1×10⁹/mL and used as 5% in medium (the values therefore have to be multiplied by at least 20). <: below the detection limit. Values with a black background are anti-inflammatory cytokines and cells with a gray background are inflammatory cytokines.

TABLE 5
Determination of factor-concentrations in PL.

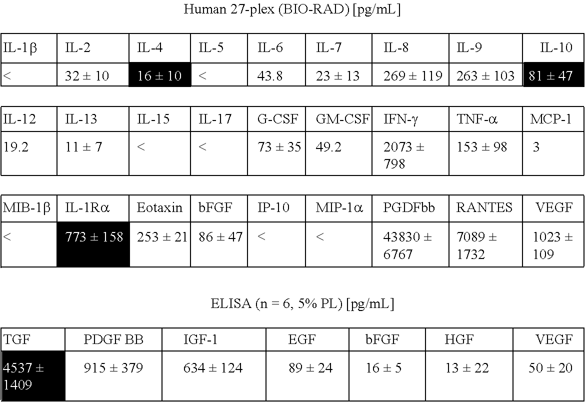

For effective expansion of MSC, an optimized preparation of PL is needed. The protocol includes pooling PRPs from at least 10 donors (to equalize for differences in cytokine concentrations) with a minimal concentration of 3×10⁹ thrombocytes/mL.

PL was prepared either from pooled platelet concentrates designed for human use or from 7-13 pooled buffy coats after centrifugation at 200×g for 20 min. PRP was aliquoted into small portions, frozen at −80° C., thus producing PL which is thawed immediately before use. PL-containing medium was prepared fresh for each cell feeding. Medium contained αMEM as basic medium supplemented with 5 IU Heparin/mL medium (Ratiopharm) and 5% of freshly thawed PL.

### Example 6

### Production of MSC in PL-Supplemented Media

Bone marrow was collected from non-mobilized healthy donors. White blood cells (WBC) concentrations and CFU-F (colony forming units-fibroblasts) from bone marrow isolated from different donors varied.

Donors were tested for infectious agents prior to donation. Testing included human immunodeficiency virus, type 1 and 2 (HIV I/II), human T cell lymphotrophic virus, type I and II (HTLV I/II), hepatitis B virus (HBV), hepatitis C virus (HCV), Treponema pallidum (syphilis) and cytomegalovirus (CMV).

25 mL-125 mL (e.g., 25, 30, 35, 40, 45, 50, 55, 60, 65, 70, 75, 80, 85, 90, 100, 105, 110, 115, 120, 125 mL) whole bone marrow was plated in αMEM media containing 2-10% (e.g., 2%, 3%, 4%, 5%, 6%, 7%, 8%, 9%, or 10%) PL in a multi layered cell factory for 2-10 days (e.g., 2, 3, 4, 5, 6, 7, 8, 9, or 10 days) to allow the MSCs to adhere. Residual non-adherent cells were washed from the cell factory. αMEM media containing 2-10% (e.g., 2%, 3%, 4%, 5%, 6%, 7%, 8%, 9%, or 10%) PL was added to the factory. Cells were allowed to grow until 70%-100% colony confluence (e.g., 70, 75, 80, 85, 90, 95, or 100%) and/or 5-15% (e.g., 5, 6, 7, 8, 9, 10, 11, 12, 13, 14, or 15%) overall surface confluence (approximately 3-33 days) with medium exchange every 4-5 days. Cells were washed with phosphate buffered saline (PBS), then detached with recombinant trypsin and re-plated into a cell factory. Cells remained in the cell factory for 6-8 days for expansion with media exchange on day 5 until they reach 80-100% surface confluence (e.g., 80, 85, 90, 95, or 100%) before they are harvested.

The cells were harvested by treating with trypsin (e.g., recombinant) and then neutralized with a stopwash solution containing 0.5-5% HSA (e.g., 0.5, 1, 1.5, 2, 2.5, 3, 3.5, 4, 4.5, or 5%) and were then aliquotted at 1 mL (about 10 million cells) per vial, then cryopreserved in 2-10% (e.g., 2, 3, 4, 5, 6, 7, 8, 9, or 10%) DMSO, 2-10% (e.g., 2, 3, 4, 5, 6, 7, 8, 9, or 10%) HSA in PlasmaLyte A using controlled-rate freezing. The cell-containing vials were stored at −130° C. or lower in vapor phase liquid nitrogen. Cell product was tested for infectious agents using methods routine in the art. Testing included human immunodeficiency virus, type 1 and 2 (HIV I/II), human T cell lymphotrophic virus, type I and II (HTLV I/II), hepatitis B virus (HBV), hepatitis C virus (HCV), Treponema pallidum (syphilis) and cytomegalovirus (CMV).

The cell-containing vials were expanded for 2 or 3 additional rounds in cell factories using a closed system. Cells were detached with trypsin (e.g., recombinate) as described above and final harvested cell product is concentrated and washed using a closed system TFF or closed system centrifugation before the cells were formulated in PlasmaLyteA, 2-10% DMSO (e.g., 2, 3, 4, 5, 6, 7, 8, 9, or 10%), and 2-10% HSA (e.g., 2, 3, 4, 5, 6, 7, 8, 9, or 10%). The final product was cryopreserved using a controlled-rate freezer and stored at −130° C. or lower in vapor phase liquid nitrogen.

When the cells were required for infusion, they were thawed and suspended in PlasmaLyte A containing 2-10% (e.g., 2, 3, 4, 5, 6, 7, 8, 9, or 10%) HSA.

The final cell product consisted of approximately 10⁶-10⁸ cells per kg of weight of the subject (depending on the dose schedule) suspended in a sufficient volume of PlasmaLyte A with 2-10% (e.g., 2, 3, 4, 5, 6, 7, 8, 9, or 10%) HSA. No growth factors, antibodies, stimulants, or any other substances were added to the product at any time during manufacturing. The final concentration was adjusted to provide the required dose such that the volume of product that is returned to the patient remained constant.

### Example 7

### Comparison of MSCs Grown in PL- and FBS-Supplemented Media

The expansion of MSCs from bone marrow (BM) has been shown to be more effective with PL-compared to FBS-supplemented media. The size, as well as the number (Table 6), of CFU-F were considerably higher using PL as supplement in the medium (see WO2010/017216 or US20110293576, incorporated herein by reference).

TABLE 6
CFU-F from MSCs with FBS- or PL-supplemented media.
Values are shown for 10⁷ plated cells.

| | αMEM + FBS | αMEM + PL |
|---|---|---|
| mean ± SE | 415 ± 97 | 1181 ± 244 |

MSCs were isolated by plating 5×10⁵ mononuclear cells/well in 3 mL. The more effective isolation of MSCs with PL-supplemented media is followed by a more rapid expansion of these cells over the whole cultivation period until senescence.

Also, MSCs cultured in PL-supplemented media are less adipogenic in character when compared to MSCs cultured in FBS-supplemented media.

MSC have been described to act in an immunomodulatory fashion by impairing T-cell activation without inducing anergy. A dilution of this effect has been shown in vitro in mixed lymphocyte cultures (MLC) leading eventually to an activation of T-cells if decreasing amounts of MSC are added to the MLC reaction. This activation process is not observed when PL-generated MSC are used in the MLC as the third party. MSCs are less immunogenic after PL-expansion whereas FBS seems to act as a strong antigen or at least has adjuvant function in T-cell stimulation. This result is also reflected in differential gene expression showing a down-regulation of MHC II compounds.

Additional data from differential gene expression analysis of PL-generated compared to FBS-generated MSC showed an up-regulation of genes involved in the cell cycle (e.g. cyclins and cyclin dependent kinases) and the DNA replication and purine metabolism. On the other hand, genes functionally active in cell adhesion/extracellular matrix (ECM)-receptor interaction, differentiation/development, TGF-β signaling and thrombospondin induced apoptosis could be shown to be downregulated in PL-generated MSC, further supporting the results of faster growth and accelerated expansion.

Furthermore, evidence demonstrates that MSCs grown in PL-supplemented medium are more protective against ischemia-reperfusion damage than MSCs grown in FBS-supplemented medium. Human kidney proximal tubular cells (HK-2) were forced to start apoptotic events by incubation with antimycin A, 2-deoxyclucose and calcium ionophore A23187 (Lee et al., J Am Soc Nephrol 13, 2753-2761 (2002); Xie et al., J Am Soc Nephrol 17, 3336-3346 (2006)). This treatment chemically mimics an ischemic event. Reperfusion was simulated by refeeding the HK-2 cells with rescue media consisting of conditioned medium incubated for 24 h on confluent layers of MSCs grown with either αMEM+10% FBS or αMEM+5% PL.

Supernatants from MSCs grown in PL-containing medium are more effective in reducing HK-2 cell death after chemically simulated ischemia/reperfusion than supernatants from MSCs grown in FBS-supplemented medium.

A parallel FACS assay detecting annexin V that binds to apoptotic cells showed similar results. The proportion of viable cells (=annexin V negative) was higher in the HK-2 cells rescued with MSC-conditioned PL medium (85.7%, as compared to 78.0% in MSC-conditioned FBS medium. Thus, it appears that PL-MSCs contain a higher rate of factors that prevent kidney tubular cells from dying after ischemic events and/or less factors that promote cell death compared to FBS-MSC conditioned medium. Thus, PL appears to be the supplement of choice to expand MSCs for the clinical treatment of ischemic injury.

### Example 8

### Safety of rMSC Administration at High Doses

In a 30-day study, AKI was induced by I/R in 9 female Sprague-Dawley rats. Rats with AKI received doses of rMSC of 5×10⁶, 10×10⁶, or 15×10⁶ rMSC per kg body weight by intra-arterial (IA) infusion. The highest dose was 15 million rMSC/kg IA. Kidney function, as measured by SCr and BUN, was determined on days 1 and 7 after infusion. Animals were euthanized 30 days after rMSC infusion, and renal histopathology was assessed. No deaths occurred in this study. SCr and BUN values were within the expected ranges after I/R-induced AKI, and there was no evidence of deleterious consequences of rMSC administration on renal function. Kidney histopathology of samples collected 30 days after rMSC administration was normal in all animals. This study supports the safety of rMSC administration via intra-arterial infusion in the setting of AKI at high doses.

### REFERENCES

Shanley et al., “Topography of focal proximal tubular necrosis after ischemia with reflow in the rat kidney”, American Journal of Pathology, 122:462-68 (1986).
Vogt et al., “On the molecular pathology of ischemic renal cell death: reversible and irreversible cellular and mitochondrial metabolic alterations,” American Journal of Pathology 53:1-26 (1968).
Tögel et al., “Administered mesenchymal stem cells protect against ischemic acute renal failure through differentiation-independent mechanisms,” Am J Physiol Renal Physiol 289:F31-42 (2005).
Zarjou et al., “Paracrine effects of mesenchymal stem cells in cisplatin-induced renal injury require heme oxygenase-1,” Am J Physiol Renal Physiol 300:F254-62 (2011).
Qian et al., “Bone marrow mesenchymal stem cells ameliorate rat acute renal failure by differentiation into renal tubular epithelial-like cells,” Int. J. Mol. Med. 22:325-332 (2008)

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method of treating acute kidney injury (AKI) in a subject comprising administering a therapeutically effective amount of human mesenchymal stem cells (hMSC) to a subject in need thereof up to at least 48 hours following a decline in kidney function in the subject, wherein the decline in kidney function is measured by an increase in serum creatinine level of at least 0.3 mg/dL, and wherein the hMSCs ameliorate AKI in the subject.

2. The method of claim 1, wherein the decline in kidney function is measured by an increase in serum creatinine level of at least 0.5 mg/dL.

3. The method of claim 1, wherein the decline in kidney function is determined by an increase in serum creatinine level of between 0.3 mg/dL and 0.5 mg/dL.

4. The method of claim 1, wherein the decline in kidney function is further measured by an increase in one or more additional serum/blood biomarkers, one or more urine biomarkers, or both.

5. The method of claim 4, wherein the one or more additional serum/blood biomarkers are selected from the group consisting of blood urea nitrogen (BUN), Cystatin C, and Beta-trace protein (BTP).

6. The method of claim 4, wherein the one or more urine biomarkers are selected from the group consisting of Podocalyxin, Nephrin, Alpha 1-microglobulin, Beta 2-microglobulin, Glutathione S-transferase, Interleukin-18, Kidney Injury Molecule-1 (KIM-1), Liver-Type Fatty Acid-Binding Protein, Netrin-1, Neutrophil Gelatinase-Associated Lipocalcin (NGAL), and N-Acetyl-Beta-D-Glucosaminidase (NAG).

7. The method of claim 1, wherein the decline in kidney function is further measured by an increase in one or more biomarkers selected from the group consisting of blood urea nitrogen (BUN), Cystatin C, Beta-trace protein (BTP), Podocalyxin, Nephrin, Alpha 1-microglobulin, Beta 2-microglobulin, Glutathione S-transferase, Interleukin-18, Kidney Injury Molecule-1 (KIM-1), Liver-Type Fatty Acid-Binding Protein, Netrin-1, Neutrophil Gelatinase-Associated Lipocalcin (NGAL), and N-Acetyl-Beta-D-Glucosaminidase (NAG).

8. The method of claim 1, wherein the therapeutically effective amount of hMSCs is between about 7×10⁵ and about 7×10⁶ cells/kg.

9. The method of claim 8, wherein the therapeutically effective amount of hMSCs is between about 2×10⁶ cells/kg and about 5×10⁶ cells/kg.

10. The method of claim 9, wherein the therapeutically effective amount of hMSCs is 2×10⁶ cells/kg.

11. The method of claim 1, wherein the hMSCs are administered to the subject at the onset of the decline in kidney function.

12. The method of claim 1, wherein the hMSCs are administered to the subject at least 24 hours following the decline in kidney function.

13. The method of claim 1, wherein the hMSCs are administered to the subject at least 48 hours following the decline in kidney function.

14. The method of claim 1, wherein the hMSCs are administered to the subject between the onset of the decline in kidney function and 24 hours following the decline in kidney function.

15. The method of claim 1, wherein the hMSCs are administered to the subject between 24 and 48 hours following the decline in kidney function.

16. The method of claim 1, wherein the hMSCs are administered intra-arterially or intravenously to the subject.

17. The method of claim 1, wherein the hMSCs are administered in a biologically and physiologically compatible solution.

18. The method of claim 17, wherein the solution is not enriched for human pluripotent hematopoietic stem cells.

19. The method of claim 1 wherein the hMSCs comprise autologous cells.

20. The method of claim 1 wherein the hMSCs comprise allogeneic cells.

21. The method of claim 1 wherein the hMSCs comprise non-transformed stem cells.

22. The method of claim 1, wherein the hMSCs are isolated from bone marrow aspirates and adhere to a culture dish while substantially all other cell types remain in suspension.

23. The method of claim 1, wherein the hMSCs are obtained from a bone marrow sample.

24. The method of claim 1, wherein the hMSCs are obtained from a cryopreserved sample.

25. The method of claim 1, wherein the hMSCs are obtained from a Master Cell Bank (MCB).

26. The method of claim 1, wherein the hMSCs are expanded in vitro to produce an enriched population of hMSCs.

27. The method of claim 26, wherein the hMSCs are expanded in a platelet lysate (PL)-supplemented culture medium.

28. The method of claim 1, wherein the hMSCs have 32 or fewer GT repeats in both alleles of the human heme oxygenase (HO-1) promoter region.

29. The method of claim 1, wherein the hMSCs have two short alleles, two medium alleles, or one short and one medium allele in the HO-1 promoter region wherein a short allele has ≦26 GT repeats in the HO-1 promoter region and wherein a medium allele has between 27 and 32 GT repeats in the HO-1 promoter region.

30. The method of claim 1, wherein the hMSCs do not have any long alleles, wherein a long allele has >32 GT repeats in the HO-1 promoter region.

31. The method of claim 1, wherein the hMSCs are genetically modified, to augment the renoprotective potency of said prior to administration to the subject.

32. The method of claim 1, wherein the method further comprises delivering a therapeutic amount of a stimulant of hMSC mobilization to the subject, wherein the stimulant mobilizes stem cells to the kidney.

33. The method of claim 1, wherein the subject suffers from or is at high risk of suffering from an acute deterioration in kidney function.

34. The method of claim 1, wherein the subject has undergone cardiac surgery.

35. The method of claim 34, wherein the decline in kidney function in the subject occurs 48 hours or less following the cardiac surgery.
